(19) United States
(12) Patent Application Publication
(10) Pub. No.: US 2025/0025076 A1
(43) Pub. Date: Jan. 23, 2025

(54) APTAMER PROTECTIVE MATERIAL AND BIOSENSOR

(71) Applicant: DexCom, Inc., San Diego, CA (US)

(72) Inventors: Shuyu Lin, San Diego, CA (US); Daiting Rong, San Diego, CA (US); Stacy Hunt DuVall, San Diego, CA (US); Wenjie Lan, San Diego, CA (US); Jason M. Halac, San Diego, CA (US); Jiong Zou, San Diego, CA (US); Devon M. Headen, San Diego, CA (US); Shanger Wang, San Diego, CA (US); Shane Richard Parnell, San Diego, CA (US); Berta Esteban Fernandez de Avila, San Diego, CA (US); Shannon Reuben Woodruff, San Diego, CA (US)

(73) Assignee: DexCom, Inc., San Diego, CA (US)

(21) Appl. No.: 18/399,162

(22) Filed: Dec. 28, 2023

Related U.S. Application Data
(60) Provisional application No. 63/436,056, filed on Dec. 29, 2022.

(51) Int. Cl. A61B 5/1473

(52) U.S. Cl. CPC A61B 5/1473

(57) ABSTRACT

An analyte monitoring sensor configured for in vivo measurement of at least one analyte is provided, the sensor comprising a substrate having a substrate surface, an aptamer protective layer encapsulating at least a portion of the substrate surface, the aptamer protective layer permeable to the at least one analyte, one or more aptamer conjugates associated with at least a portion of the substrate surface and positioned between the aptamer protective layer and the substrate for obtaining measurements related to the at least one analyte in vivo. Methods of extending in vivo performance of the analyte monitoring sensor are also described.

### TECHNICAL FIELD

This disclosure is directed to protective materials for an aptamer-based biosensor construct or device suitable for wholly or partially implantation in a subject for continuous monitoring of an analyte.

### BACKGROUND

Aptamer biosensors (AB) are a class of affinity biosensors in which the recognition element is the aptamer (single stranded DNA/RNA) which has a specific affinity to the analyte, and such aptamer-analyte interaction induces a measurable transduced signal (optical, electrical). Existing aptamer biosensors (AB's) are limited to the poor stability observed when placed in physiological relevant environments, which in part is at least attributed to desorption of the aptamer monolayer from a substrate surface or desorption of the underneath monolayer used to immobilize the aptamer. These desorption events limit the application of AB's for continuous analyte monitoring in physiological environments. Another weakness of AB sensors, specifically, electrochemical aptamer biosensors (EAB's) is that their bioelectronic interface degrades upon continuous electrochemical interrogation and/or biofouling—a process typically seen as a drop in faradaic and an increase in charging currents over time. This progressive degradation limits EAB's in vivo operational life to 12 hours or less, a period that is much shorter than the elimination half-life of the vast majority of drugs in humans.

### SUMMARY

In a first example, an analyte monitoring sensor configured for in vivo measurement of at least one analyte is provided, comprising: a substrate having a substrate surface; an aptamer protective layer encapsulating at least a portion of the substrate surface, the aptamer protective layer permeable to the at least one analyte; one or more aptamer conjugates associated with at least a portion of the substrate surface and positioned between the aptamer protective layer and the substrate for obtaining measurements related to the at least one analyte in vivo; and a reversible redox moiety coupled to the one or more aptamer conjugates.

In one aspect, alone or in combination with any one of the previous aspects, at least a portion of the substrate is a conductive metal. In one aspect, alone or in combination with any one of the previous aspects, at least a portion of the substrate is gold, carbon, graphene, or graphene oxide. In one aspect, alone or in combination with any one of the previous aspects, at least a portion of the substrate comprises pores having average pore diameters with nanometer and/or micrometer dimensions.

In one aspect, alone or in combination with any one of the previous aspects, at least a portion of the substrate surface further comprises one or more co-adsorbents. In one aspect, alone or in combination with any one of the previous aspects, the one or more co-adsorbents independently comprises a plurality of functional groups.

In one aspect, alone or in combination with any one of the previous aspects, the one or more co-adsorbents provide, independently, for one or more of a surface energy range, a phase separation range, and an intermolecular interaction range between the one or more aptamers and the aptamer protective layer. In one aspect, alone or in combination with any one of the previous aspects, the one or more co-adsorbents provide, independently, an ionic strength, a pH range, or pH buffering, and an amount of the one or more co-adsorbents is present capable of modulating or maintaining the ionic strength, the pH range, or pH buffering in proximity to the at least one aptamer conjugates.

In one aspect, alone or in combination with any one of the previous aspects, at least a portion of the substrate surface the one or more co-adsorbents and a portion of a remainder of the substrate surface comprises the one or more aptamer conjugates. In one aspect, alone or in combination with any one of the previous aspects, at least a portion of the substrate surface comprises the one or more co-adsorbents and a portion of a remainder of the substrate surface comprises the one or more aptamer conjugates physically or chemically coupled thereto. In one aspect, alone or in combination with any one of the previous aspects, the one or more co-adsorbents are physically or chemically coupled to the substrate surface and a portion of a remainder of the substrate surface comprises the one or more aptamer conjugates physically or chemically coupled thereto.

In one aspect, alone or in combination with any one of the previous aspects, the co-adsorbate comprises a self-assembled monolayer (SAM). In one aspect, alone or in combination with any one of the previous aspects, the co-adsorbate coupled or tethered to a substrate is represented as follows:

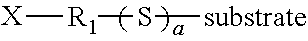

where X is —OH, —NHR1, —NH2 or —SH; where R1 is acyclic alkyl, substituted or unsubstituted cyclic alkyl, substituted or unsubstituted aryl, substituted or unsubstituted benzyl, substituted or unsubstituted heteroalkyl, or substituted or unsubstituted heterocyclic; and a is 1-3.

In one aspect, alone or in combination with any one of the previous aspects, the co-adsorbate comprises a mono-functional or a multi-functional alkanethiol, hydroxyalkyl mercaptan, alkoxymercaptan, alkylarylthiol, hydroxyalkylarylthiol, hydroxylalkylarylmercaptan, alkylarylmercaptoalkanol, alkylmercaptophenol, alkylmercaptocatechol, arylmercaptophenol, arylmercaptocatechol, alkoxyarylthiol, alkoxyarylmercaptan (collectively used hereinafter as “thiol-co-adsorbent”). In one aspect, alone or in combination with any one of the previous aspects, a thiol functional group of the mono-functional alkanethiol or the multi-functional alkanethiol is covalently coupled to at least a portion of the substrate surface.

In one aspect, alone or in combination with any one of the previous aspects, a thiol-co-adsorbent is covalently coupled to a gold substrate surface. In one aspect, alone or in combination with any one of the previous aspects, the co-adsorbate comprises a mono-functional or a multi-functional mercaptoalkanol, benzylmercaptoalkanol, or arylmercaptoalkanol (collectively used hereinafter as “(aryl)mercaptoalkanol”). In one aspect, alone or in combination with any one of the previous aspects, a thiol functional group of the mono-functional or the multi-functional (aryl)mercaptoalkanol is covalently coupled to at least a portion of the substrate surface. In one aspect, alone or in combination with any one of the previous aspects, a thiol functional group of the mono-functional or the multi-functional (aryl)mercaptoalkanol is covalently coupled to at least a portion of a gold substrate surface.

In one aspect, alone or in combination with any one of the previous aspects, at least a portion of the substrate surface comprises a zwitterionic repeating group. In one aspect, alone or in combination with any one of the previous aspects, the zwitterionic repeating group comprises a betaine group. In one aspect, alone or in combination with any one of the previous aspects, the zwitterionic repeating group comprises ammoniophosphates or lecithin analogs.

In one aspect, alone or in combination with any one of the previous aspects, the zwitterionic repeating group comprises ammoniophosphonates. In one aspect, alone or in combination with any one of the previous aspects, the zwitterionic repeating group comprises ammoniophosphinates. In one aspect, alone or in combination with any one of the previous aspects, the zwitterionic repeating group comprises ammoniosulfonates. In one aspect, alone or in combination with any one of the previous aspects, the zwitterionic repeating group comprises ammoniosulfates. In one aspect, alone or in combination with any one of the previous aspects, the zwitterionic repeating group comprises ammoniocarboxylates.

In one aspect, alone or in combination with any one of the previous aspects, the zwitterionic repeating group comprises an alkanethiol betaine, phenylthiol betaine, or benzylthiol betaine. In one aspect, alone or in combination with any one of the previous aspects, the alkanethiol, phenylthiol, or benzylthiol is linear and comprises a plurality of betaine groups along its chain. In one aspect, alone or in combination with any one of the previous aspects, the alkanethiol, phenylthiol, or benzylthiol is an end-terminated mono- or di-thiol with at least one betaine groups along its chain or aromatic ring. In one aspect, alone or in combination with any one of the previous aspects, the zwitterionic repeating group comprises n-mercaptoalkanol betaine. In one aspect, alone or in combination with any one of the previous aspects, the mercaptoalkanol is linear and comprises a plurality of betaine groups along its chain. In one aspect, alone or in combination with any one of the previous aspects, the mercaptophenol comprises one or more betaine groups attached to an aromatic ring. In one aspect, alone or in combination with any one of the previous aspects, the mercaptoalkanol or mercaptophenol is a 1,2-dithiol, 1,3-dithiol or 1,4-dithiol of an alkyl or aromatic hydrocarbon compound.

In one aspect, alone or in combination with any one of the previous aspects, a thiol group of the end-terminated dithiol alkanethiol is covalently coupled to the substrate surface. In one aspect, alone or in combination with any one of the previous aspects, a thiol group of the mercaptoalkanol is covalently coupled to the substrate surface.

In one aspect, alone or in combination with any one of the previous aspects, the co-adsorbent is selected from one or more of the following structures:

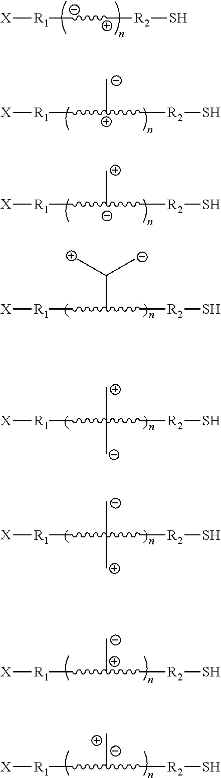

where “ [symbol] ” represents a hydrocarbon chain; where R1 and R2 are, independently, branched or unbranched acyclic alkyl, substituted or unsubstituted cyclic alkyl, substituted or unsubstituted aryl, substituted or unsubstituted benzyl, substituted or unsubstituted heteroalkyl, substituted or unsubstituted heterocyclic; where X is —OH, —NHR1, —NH2, or —SH; where n is an integer of 2 to about 1000; or

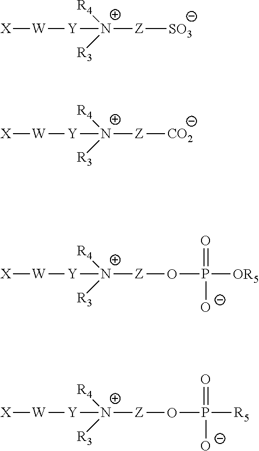

where X is —OH, —NHR1, —NH2, or —SH; where W, Y, and Z are, independently, branched or straight chain alkyl, heteroalkyl, cycloalkyl, cycloheteroalkyl, aryl, or heteroaryl, any of which can be optionally substituted with O, OH, halogen, amido, or alkoxyl; R1 is H, branched or unbranched acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl; and R3, R4, and R5, are independently chosen from acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl. In one example, one or more of R1, R2, R3, R4, R5,and Z are covalently or ionically coupled to the APL.

In one aspect, alone or in combination with any one of the previous aspects, at least a portion of the substrate surface comprises covalently coupled aliphatic amine. In one aspect, alone or in combination with any one of the previous aspects, at least a portion of the substrate surface comprises covalently coupled amino alkanoic acid.

In one aspect, alone or in combination with any one of the previous aspects, at least a portion of a carbon, a graphene, or a graphene oxide substrate surface comprises covalently coupled amino alkanoic acid. In one aspect, alone or in combination with any one of the previous aspects, at least a portion of a carbon, a graphene, or a graphene oxide substrate surface comprises covalently coupled amino alkanoic acid and the amino alkanoic acid is also covalently coupled to the one or more aptamer conjugates.

In one aspect, alone or in combination with any one of the previous aspects, the aptamer protective layer is at least partially cross-linked using an amount of cross-linking agent. In one aspect, alone or in combination with any one of the previous aspects, the aptamer protective layer comprises a conductive polymer. In one aspect, alone or in combination with any one of the previous aspects, the aptamer protective layer comprises a zwitterionic group compound. In one aspect, alone or in combination with any one of the previous aspects, the aptamer protective layer comprises a zwitterionic repeating group compound.

In one aspect, alone or in combination with any one of the previous aspects, the aptamer protective layer provides an ionic strength, a pH range, or pH buffering. In one aspect, alone or in combination with any one of the previous aspects, the aptamer protective layer provides an amount of a zwitterionic repeating group compound is present capable of modulating or maintaining an ionic strength, a pH range, or pH buffering. In one aspect, alone or in combination with any one of the previous aspects, the aptamer protective layer provides a free volume allowing reversible conformational change of the one or more aptamer conjugates present therein, the free volume sufficient to provide a signal in the presence of the at least one analyte.

In one aspect, alone or in combination with any one of the previous aspects, the aptamer protective layer comprises a functionalized polymer. In one aspect, alone or in combination with any one of the previous aspects, the functionalized polymer comprises alkanethiol groups. In one aspect, alone or in combination with any one of the previous aspects, the alkanethiol groups are present at the end of the functionalized polymer chain. In one aspect, alone or in combination with any one of the previous aspects, the alkanethiol groups are present along the backbone of the functionalized polymer chain.

In one aspect, alone or in combination with any one of the previous aspects, the functionalized polymer comprises mercaptoalkanol groups. In one aspect, alone or in combination with any one of the previous aspects, the mercaptoalkanol groups are present at the end of the functionalized polymer chain. In one aspect, alone or in combination with any one of the previous aspects, the mercaptoalkanol groups are present along the backbone of the functionalized polymer chain.

In one aspect, alone or in combination with any one of the previous aspects, the aptamer protective layer comprises a functionalized polymer comprising one or more zwitterionic repeating groups. In one aspect, alone or in combination with any one of the previous aspects, the one or more zwitterionic repeating groups comprise a betaine compound or derivative thereof. In one aspect, alone or in combination with any one of the previous aspects, the zwitterionic repeating groups are present at the end of the functionalized polymer chain. In one aspect, alone or in combination with any one of the previous aspects, the zwitterionic repeating groups are present along the backbone of the functionalized polymer chain.

In one aspect, alone or in combination with any one of the previous aspects, the functionalized polymer comprises alkanethiol and zwitterionic repeating groups. In one aspect, alone or in combination with any one of the previous aspects, the functionalized polymer comprises alkanethiol and betaine groups. In one aspect, alone or in combination with any one of the previous aspects, the functionalized polymer comprises mercaptoalkanol and zwitterionic repeating groups.

In one aspect, alone or in combination with any one of the previous aspects, the functionalized polymer comprises mercaptoalkanol and betaine groups.

In one aspect, alone or in combination with any one of the previous aspects, the aptamer protective layer is physically or chemically coupled to at least a portion of the substrate surface. In one aspect, alone or in combination with any one of the previous aspects, the aptamer protective layer is physically or chemically coupled to at least a portion of the substrate surface, the one or more aptamer conjugates are physically or chemically coupled to at least a portion of the substrate surface, and a substantial remainder of the substrate surface further comprises a physically or chemically coupled co-adsorbate.

In one aspect, alone or in combination with any one of the previous aspects, the aptamer protective layer comprises at least one polymer segment selected from the group consisting of polyurethane, polyurea, poly(urethane urea), epoxide, polyolefin, polysiloxane, polyamide, polystyrene, polyacrylate, polyether, polyvinylpyridine, polyvinylpyrrolidone, polyester, polycarbonate, and copolymers thereof.

In one aspect, alone or in combination with any one of the previous aspects, the aptamer protective layer comprises a segmented multiblock polymer. In one aspect, alone or in combination with any one of the previous aspects, the aptamer protective layer comprises a segmented multiblock polyurethane polymer. In one aspect, alone or in combination with any one of the previous aspects, the aptamer protective layer comprises a segmented multiblock polyurethane urea polymer.

In one aspect, alone or in combination with any one of the previous aspects, the segmented multiblock polymer comprises a soft segment and a hard segment. In one aspect, alone or in combination with any one of the previous aspects, the soft segment is hydrophobic or hydrophilic. In one aspect, alone or in combination with any one of the previous aspects, the soft segment is hydrophobic and hydrophilic. In one aspect, alone or in combination with any one of the previous aspects, the soft segment comprises hydrophobic polyol and hydrophilic polyol.

In one aspect, alone or in combination with any one of the previous aspects, the soft segment is one or more segments comprising polydimethylsiloxane, polycarbonate, polyester, polyether, and blends or copolymers thereof. In one aspect, alone or in combination with any one of the previous aspects, the soft segment is one or more segments comprising polyethylene glycol, oligo polyether, polyoxazoline (POX), polypeptide, polyvinylpyrrolidone, zwitterionic repeating group polymer, and blends or copolymers thereof.

In one aspect, alone or in combination with any one of the previous aspects, the hard segment comprises urethane groups or urea groups.

In one aspect, alone or in combination with any one of the previous aspects, the one or more aptamer conjugates is physically associated to a portion of the substrate surface. In one aspect, alone or in combination with any one of the previous aspects, the one or more aptamer conjugates covalently associated to a portion of the substrate surface.

In one aspect, alone or in combination with any one of the previous aspects, the one or more aptamer conjugates comprises RNA or DNA nucleotide sequences. In one aspect, alone or in combination with any one of the previous aspects, the one or more aptamer conjugates comprises at least one of: 2′-O-methyl modification of a nucleotide; disulfide bridges; a 3′ cap with an inverted 2-deoxy thymidine; a 3′-3′-thymidine linkage at 3′ terminus; a 2′-F modification; and a double stranded section. In one aspect, alone or in combination with any one of the previous aspects, the one or more aptamer conjugates comprises RNA or DNA sequences with a first linker moiety on a 5′ end and the reversible redox moiety at a 3′ end. In one aspect, alone or in combination with any one of the previous aspects, the one or more aptamer conjugates comprises RNA or DNA sequences with a first linker moiety on a 3′ end and the reversible redox moiety at a 5′ end.

In one aspect, alone or in combination with any one of the previous aspects, the first linker moiety on the 5′ end or the 3′ end of the aptamer comprises an amino group or carboxyl group. In one aspect, alone or in combination with any one of the previous aspects, the first linker moiety is physically or chemically coupled to the substrate at the 5′ end. In one aspect, alone or in combination with any one of the previous aspects, the first linker moiety is physically or chemically coupled to the co-adsorbate at the 5′ end. In one aspect, alone or in combination with any one of the previous aspects, the first linker moiety is physically or chemically coupled to the substrate at the 3′ end. In one aspect, alone or in combination with any one of the previous aspects, the first linker moiety is physically or chemically coupled to the co-adsorbate at the 3′ end.

In one aspect, alone or in combination with any one of the previous aspects, the one or more aptamer conjugates is a glycopeptide antibiotic binding aptamer. In one aspect, alone or in combination with any one of the previous aspects, the one or more aptamer conjugates is a vancomycin binding aptamer. In one aspect, alone or in combination with any one of the previous aspects, the one or more aptamer conjugates is a neurotransmitter binding aptamer. In one aspect, alone or in combination with any one of the previous aspects, the one or more aptamer conjugates is a dopamine or glutamate binding aptamer. In one aspect, alone or in combination with any one of the previous aspects, the one or more aptamer conjugates is a carbohydrate, triglyceride or fatty acid binding aptamer. In one aspect, alone or in combination with any one of the previous aspects, the one or more aptamer conjugates is a glucose, glycerol, or beta-hydroxybutyrate binding aptamer.

In one aspect, alone or in combination with any one of the previous aspects, the one or more aptamer conjugates is physically or chemically coupled to a self-assembled monolayer (SAM). In one aspect, alone or in combination with any one of the previous aspects, the one or more aptamer conjugates is physically or chemically coupled to a mono-functional or multi-functional alkanethiol or mercaptoalkanol. In one aspect, alone or in combination with any one of the previous aspects, the one or more aptamer conjugates is physically or chemically coupled to an alkylthiol betaine.

In one aspect, alone or in combination with any one of the previous aspects, the one or more aptamer conjugates is physically or chemically coupled to an aliphatic amine. In one aspect, alone or in combination with any one of the previous aspects, the one or more aptamer conjugates is physically or chemically coupled to an amino alkanoic acid.

In one aspect, alone or in combination with any one of the previous aspects, the reversible redox moiety comprises iron, iridium, ruthenium, osmium, a thiazine dye, or derivative thereof. In one aspect, alone or in combination with any one of the previous aspects, the reversible redox moiety comprises ferrocene or methylene blue.

In one aspect, alone or in combination with any one of the previous aspects, the sensor is configured for continuous, semi-continuous, sequential, or random signal acquisition. In one aspect, alone or in combination with any one of the previous aspects, the sensor further comprises one or more of a reference electrode, a working electrode and a counter electrode. In one aspect, alone or in combination with any one of the previous aspects, the sensor further comprises one or more of a transmitter, receiver, controller, or power supply. In one aspect, alone or in combination with any one of the previous aspects, the sensor is configured for transcutaneous insertion.

In a second example, a method of extending end of life of an electrochemical aptamer biosensor (EAB) is provided, the method comprising: electrically associating at least one aptamer conjugate to a surface of a conductive substrate, the at least one aptamer conjugate comprising a reversible redox moiety; encapsulating the at least one aptamer conjugate in an aptamer protective layer, the at least one aptamer conjugate configured to undergo a reversible confirmation change within the aptamer protective layer in response interaction with an analyte so as to generate a detectable signal; controlling one or more of: ionic strength, a pH range, or pH buffering within the aptamer protective layer, surface phase separation of the aptamer protective layer, and intermolecular interactions between the at least one aptamer conjugate and the aptamer protective layer; and extending the end-of-life of the electrochemical aptamer sensor.

In one aspect, alone or in combination with any one of the previous aspects, controlling ionic strength, providing a pH range, or pH buffering comprises introducing one or more co-adsorbents to the aptamer protective layer, the one or more co-adsorbents present in an amount capable of modulating or maintaining the ionic strength, the pH range, or pH buffering.

In one aspect, alone or in combination with any one of the previous aspects, the one or more co-adsorbents comprises a zwitterionic betaine group. In one aspect, alone or in combination with any one of the previous aspects, the one or more co-adsorbents comprising a zwitterionic betaine group is selected from the following structures:

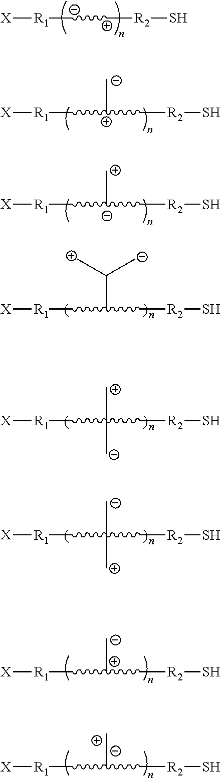

where “ [symbol] ” represents a hydrocarbon chain; where R1 and R2 are, independently, branched or unbranched acyclic alkyl, substituted or unsubstituted cyclic alkyl, substituted or unsubstituted aryl, substituted or unsubstituted benzyl, substituted or unsubstituted heteroalkyl, substituted or unsubstituted heterocyclic; where X is —OH, —NHR1, —NH2, or —SH; where n is an integer of 2 to about 1000; or

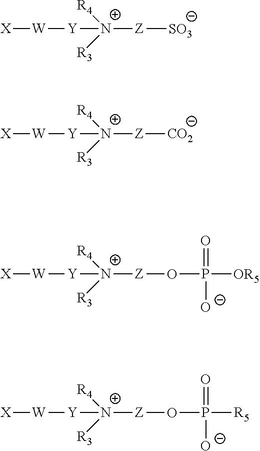

where X is —OH, —NHR1, —NH2, or —SH; where W, Y, and Z are, independently, branched or straight chain alkyl, heteroalkyl, cycloalkyl, cycloheteroalkyl, aryl, or heteroaryl, any of which can be optionally substituted with O, OH, halogen, amido, or alkoxyl; R1 is H, branched or unbranched acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl; and R3, R4, and R5, are independently chosen from acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl. In one example, one or more of R1, R2, R3, R4, R5,and Z are covalently or ionically coupled to the APL.

In one aspect, alone or in combination with any one of the previous aspects, the co-adsorbate is an end-terminated dithiol with at least one betaine groups along its chain. In one aspect, alone or in combination with any one of the previous aspects, a thiol group of the end-terminated dithiol alkanethiol is covalently coupled to the substrate surface. In one aspect, alone or in combination with any one of the previous aspects, the zwitterionic betaine group comprises an mercaptoalkanol betaine.

In one aspect, alone or in combination with any one of the previous aspects, the mercaptoalkanol is linear and comprises a plurality of betaine groups along its chain. In one aspect, alone or in combination with any one of the previous aspects, a thiol group of the mercaptoalkanol is covalently coupled to the substrate surface.

In one aspect, alone or in combination with any one of the previous aspects, controlling ionic strength a pH range, or pH buffering comprises providing the aptamer protective layer with one or more zwitterionic betaine groups. In one aspect, alone or in combination with any one of the previous aspects, controlling ionic strength comprises providing the aptamer protective layer with mercaptoalkanol and zwitterionic betaine groups. In one aspect, alone or in combination with any one of the previous aspects, the aptamer protective layer comprises alkanethiol, mercaptoalkanol, benzylthiol, mercaptophenol and one or more zwitterionic groups.

In one aspect, alone or in combination with any one of the previous aspects, controlling ionic strength a pH range, or pH buffering comprises providing the aptamer protective layer with a pH adjusting composition or pH buffering composition.

In one aspect, alone or in combination with any one of the previous aspects, controlling intermolecular interactions between the at least one aptamer conjugate and the aptamer protective layer comprises providing the aptamer protective layer with a segmented multiblock polymer backbone. In one aspect, alone or in combination with any one of the previous aspects, the segmented multiblock polymer backbone comprises a polyurethane polymer. In one aspect, alone or in combination with any one of the previous aspects, the segmented multiblock polymer backbone comprises a polyurethane urea polymer.

In one aspect, alone or in combination with any one of the previous aspects, the segmented multiblock polymer comprises a soft segment and a hard segment. In one aspect, alone or in combination with any one of the previous aspects, the soft segment is hydrophobic or hydrophilic. In one aspect, alone or in combination with any one of the previous aspects, the soft segment is hydrophobic and hydrophilic. In one aspect, alone or in combination with any one of the previous aspects, the soft segment comprises hydrophobic polyol and hydrophilic polyol. In one aspect, alone or in combination with any one of the previous aspects, the soft segment is one or more segments comprising polydimethylsiloxane, polycarbonate, polyester, polyether, and blends or copolymers thereof.

In one aspect, alone or in combination with any one of the previous aspects, the soft segment is one or more segments comprising polyethylene glycol, oligo polyether, polyoxazoline (POX), polypeptide, polyvinylpyrrolidone, polyvinylpyridine, zwitterionic repeating group polymer, and blends or copolymers thereof.

In one aspect, alone or in combination with any one of the previous aspects, the segmented multiblock polymer comprises a soft segment and a hard segment. In one aspect, alone or in combination with any one of the previous aspects, the hard segment comprises urethane groups or urea groups.

In one aspect, alone or in combination with any one of the previous aspects, the soft segment is hydrophobic or hydrophilic. In one aspect, alone or in combination with any one of the previous aspects, the soft segment is hydrophobic and hydrophilic. In one aspect, alone or in combination with any one of the previous aspects, the soft segment comprises hydrophobic polyol and hydrophilic polyol. In one aspect, alone or in combination with any one of the previous aspects, the soft segment is one or more segments comprising polydimethylsiloxane, polycarbonate, polyester, polyether, and blends or copolymers thereof. In one aspect, alone or in combination with any one of the previous aspects, the soft segment is one or more segments comprising polyethylene glycol, oligo polyether, polyoxazoline (POX), polypeptide, polyvinylpyrrolidone, zwitterionic repeating group polymer, and blends or copolymers thereof.

In one aspect, alone or in combination with any one of the previous aspects, reducing biofouling comprises providing the aptamer protective layer as defined in any one of the previous aspects.

In one aspect, alone or in combination with any one of the previous aspects, reducing decoupling of the at least one aptamer from the surface of the conductive substrate comprises coupling the at least one aptamer conjugate to a conductive substrate using carbodiimide coupling to the conductive surface.

In one aspect, alone or in combination with any one of the previous aspects, reducing oxidation of the aptamer comprises introducing one or more non-diffusible antioxidants into the aptamer protective layer.

In one aspect, alone or in combination with any one of the previous aspects, controlling diffusion of the at least one aptamer comprises at least partially cross-linking the aptamer protective layer.

In one aspect, alone or in combination with any one of the previous aspects, end-of-life is extended up to one day, 2 days, one week, 2 weeks, 3 weeks, or at least one month.

In another example, an aptamer protective layer configured for transcutaneous in vivo continuous online monitoring is provided, the aptamer protective layer comprising a polymer selected from: a functionalized polymer comprising at least one zwitterionic repeating group; a functionalized polymer derived from at least one of a polymerizable zwitterionic monomer structure as follows:

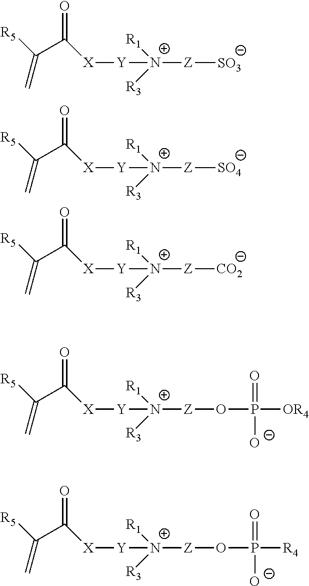

where X is O, NH, or NR4, Y and Z are, independently, acyclic alkyl, substituted or unsubstituted heteroalkyl, substituted or unsubstituted cycloalkyl, substituted or unsubstituted and of which can be optionally substituted with OH, halogen, or alkoxyl; R1, R3, R4, and R5 are independently H, alkyl, heteroalkyl, cycloalkyl, cycloheteroalkyl, aryl, or heteroaryl; a functionalized polymer comprising alkanethiol groups; a functionalized polymer comprising alkanethiol groups and zwitterionic repeating groups; a functionalized polymer comprising (aryl) mercaptoalkanol groups; a functionalized polymer comprising (aryl) mercaptoalkanol groups and zwitterionic repeating groups; or a segmented multiblock polymer.

In one aspect, alone or in combination with any one of the previous aspects, the aptamer protective layer is at least partially cross-linked.

In one aspect, alone or in combination with any one of the previous aspects, the aptamer protective layer provides an ionic strength, and an amount of a zwitterionic repeating group compound is present capable of modulating or maintaining the ionic strength. In one aspect, alone or in combination with any one of the previous aspects, the aptamer protective layer provides a free volume allowing reversible conformational change of the one or more aptamer conjugates present sufficient to provide a detectable signal in the presence of an analyte.

In one aspect, alone or in combination with any one of the previous aspects, the alkanethiol or arylthiol groups are present at the end of the functionalized polymer chain. In one aspect, alone or in combination with any one of the previous aspects, the alkanethiol or arylthiol groups are present along the backbone of the functionalized polymer chain. In one aspect, alone or in combination with any one of the previous aspects, (aryl) mercaptoalkanol groups are present at the end of the functionalized polymer chain. In one aspect, alone or in combination with any one of the previous aspects, the (aryl) mercaptoalkanol groups are present along the backbone of the functionalized polymer chain. In one aspect, alone or in combination with any one of the previous aspects, the zwitterionic repeating groups are present at the end of the functionalized polymer chain. In one aspect, alone or in combination with any one of the previous aspects, the zwitterionic repeating groups are present along the backbone of the functionalized polymer chain.

In one aspect, alone or in combination with any one of the previous aspects, the one or more zwitterionic repeating groups comprise a betaine compound or derivative thereof. In one aspect, alone or in combination with any one of the previous aspects, the aptamer protective layer is configured to physically or chemically couple to at least a portion of a substrate surface.

In one aspect, alone or in combination with any one of the previous aspects, the aptamer protective layer comprises a segmented multiblock polyurethane polymer.

In one aspect, alone or in combination with any one of the previous aspects, the segment multiblock polymer comprises at least one of polyurethane, polyurea, poly (urethane urea), epoxide, polyolefin, polysiloxane, polyamide, polystyrene, polyacrylate, polyether, polyol, polyvinylpyridine, polyvinylpyrrolidone, polyester, polycarbonate, and copolymers thereof.

In one aspect, alone or in combination with any one of the previous aspects, the aptamer protective layer comprises a segmented multiblock polyurethane urea polymer.

In one aspect, alone or in combination with any one of the previous aspects, the segmented multiblock polymer comprises a soft segment and a hard segment. In one aspect, alone or in combination with any one of the previous aspects, the soft segment is hydrophobic or hydrophilic. In one aspect, alone or in combination with any one of the previous aspects, the soft segment is hydrophobic and hydrophilic. In one aspect, alone or in combination with any one of the previous aspects, the soft segment comprises hydrophobic polyol and hydrophilic polyol.

In one aspect, alone or in combination with any one of the previous aspects, the soft segment is one or more segments comprising polydimethylsiloxane, polycarbonate, polyester, polyether, and blends or copolymers thereof. In one aspect, alone or in combination with any one of the previous aspects, the soft segment is one or more segments comprising polyethylene glycol, oligo polyether, polyoxazoline (POX), polypeptide, polyvinylpyrrolidone, polyvinylpyridine, zwitterionic repeating group polymer, and blends or copolymers thereof.

In one aspect, alone or in combination with any one of the previous aspects, the aptamer protective layer has an average molecular weight of from about 1 kDa to about 500 kDa.

In another example, a method of determining an in vivo concentration of an analyte is provided, the method comprising: contacting, in vivo, an biological fluid comprising an analyte with an electrochemical aptamer biosensor coupled to a conductive substrate, the aptamer probe encapsulated in an aptamer protective layer, the aptamer protective layer being permeable to an analyte, the electrochemical aptamer biosensor producing a signal upon interaction with the analyte; and interrogating the conductive substrate or the electrochemical aptamer; and detecting the signal corresponding to an in vivo concentration of the analyte.

In one aspect, alone or in combination with any one of the previous aspects, the interrogating is continuous, semi-continuous, sequential, or random detecting of the signal. In one aspect, alone or in combination with any one of the previous aspects, further comprising adjusting the signal based on a background signal produced as a result of non-specific binding of the aptamer biosensor so as to produce an adjusted signal.

In one aspect, alone or in combination with any one of the previous aspects, further comprising determining the in vivo concentration of the analyte during a period of time based on the adjusted signal. In one aspect, alone or in combination with any one of the previous aspects, interrogating the conductive substrate comprises a differential measurement technique.

In one aspect, alone or in combination with any one of the previous aspects, the differential measurement technique comprises interrogating the conductive substrate with a first square wave voltammetry (SWV) frequency to obtain a first signal and a second SWV frequency to obtain a second signal, taking the difference between the two signals, and dividing by the average of the two signals to obtain an adjusted signal.

In one aspect, alone or in combination with any one of the previous aspects, the interrogating comprises chronoamperometry. In one aspect, alone or in combination with any one of the previous aspects, the interrogating comprises cyclic voltammetry.

In one aspect, alone or in combination with any one of the previous aspects, the conductive substrate is an electrode, microporous or nanoporous conductive material.

In another example, a method of manufacturing an electrochemical aptamer biosensor (EAB) is provided, the method comprising: presenting at least one aptamer to at least a portion of a surface of a conductive substrate, the at least one aptamer conjugate comprising a reversible redox moiety; and presenting an aptamer protective layer to the portion of the surface of the conductive substrate; and encapsulating at least a portion of the at least one aptamer conjugate in the aptamer protective layer.

In one aspect, alone or in combination with any one of the previous aspects, further including one or more co-adsorbents into the aptamer protective layer. In one aspect, alone or in combination with any one of the previous aspects, the one or more co-adsorbents comprises a zwitterionic betaine group. In one aspect, alone or in combination with any one of the previous aspects, the one or more zwitterionic betaine groups are selected from the following structures:

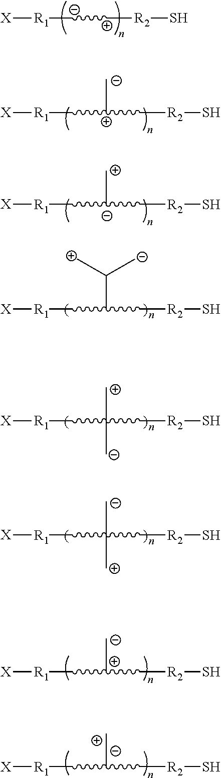

where “ [symbol] ” represents a hydrocarbon chain; where R1 and R2 are, independently, branched or unbranched acyclic alkyl, substituted or unsubstituted cyclic alkyl, substituted or unsubstituted aryl, substituted or unsubstituted benzyl, substituted or unsubstituted heteroalkyl, substituted or unsubstituted heterocyclic; where X is —OH, —NHR1, —NH2, or —SH; where n is an integer of 2 to about 1000; or

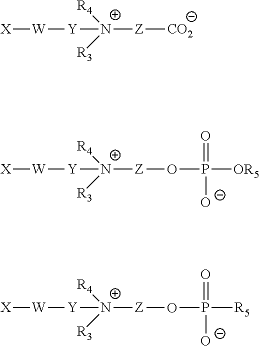

where X is —OH, —NHR1, —NH2, or —SH; where W, Y, and Z are, independently, branched or straight chain alkyl, heteroalkyl, cycloalkyl, cycloheteroalkyl, aryl, or heteroaryl, any of which can be optionally substituted with O, OH, halogen, amido, or alkoxyl; R1 is H, branched or unbranched acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl; and R3, R4, and R5, are independently chosen from acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl. In one example, one or more of R1, R2, R3, R4, R5, and Z are covalently or ionically coupled to the APL.

In one aspect, alone or in combination with any one of the previous aspects, the co-adsorbate is an end-terminated dithiol with at least one betaine groups along its chain. In one aspect, alone or in combination with any one of the previous aspects, a thiol group of the end-terminated dithiol alkanethiol is covalently coupled to the substrate surface.

In one aspect, alone or in combination with any one of the previous aspects, the zwitterionic betaine group comprises an mercaptoalkanol betaine. In one aspect, alone or in combination with any one of the previous aspects, the mercaptoalkanol is linear and comprises a plurality of betaine groups along its chain.

In one aspect, alone or in combination with any one of the previous aspects, a thiol group of the mercaptoalkanol is covalently coupled to the substrate surface.

In one aspect, alone or in combination with any one of the previous aspects, the aptamer protective layer comprises alkanethiol and one or more zwitterionic betaine groups.

In one aspect, alone or in combination with any one of the previous aspects, the aptamer protective layer comprises a segmented multiblock polymer backbone. In one aspect, alone or in combination with any one of the previous aspects, the segmented multiblock polymer backbone comprises a polyurethane polymer. In one aspect, alone or in combination with any one of the previous aspects, the segmented multiblock polymer backbone comprises a polyurethane urea polymer.

In one aspect, alone or in combination with any one of the previous aspects, the segmented multiblock polymer comprises a soft segment and a hard segment. In one aspect, alone or in combination with any one of the previous aspects, the soft segment is hydrophobic or hydrophilic. In one aspect, alone or in combination with any one of the previous aspects, the soft segment is hydrophobic and hydrophilic. In one aspect, alone or in combination with any one of the previous aspects, the soft segment comprises hydrophobic polyol and hydrophilic polyol.

In one aspect, alone or in combination with any one of the previous aspects, the soft segment is one or more segments comprising polydimethylsiloxane, polycarbonate, polyester, polyether, and blends or copolymers thereof. In one aspect, alone or in combination with any one of the previous aspects, the soft segment is one or more segments comprising polyethylene glycol, oligo polyether, polyoxazoline (POX), polypeptide, polyvinylpyrrolidone, zwitterionic repeating group polymer, and blends or copolymers thereof.

In one aspect, alone or in combination with any one of the previous aspects, the segmented multiblock polymer comprises a soft segment and a hard segment. In one aspect, alone or in combination with any one of the previous aspects, the hard segment comprises urethane groups or urea groups.

In one aspect, alone or in combination with any one of the previous aspects, the soft segment is one or more segments comprising polydimethylsiloxane, polycarbonate, polyester, polyether, and blends or copolymers thereof.

In one aspect, alone or in combination with any one of the previous aspects, the aptamer protective layer is cross-linked using an amount of cross-linking agent.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIGS. 1A and 1B are schematic diagrams illustrating an aptamer protective material employed in a biosensor in accordance with the broadest aspect of the present disclosure.

FIG. 2A is a schematic diagram illustrating an exemplary aptamer biosensor construct.

FIGS. 2B, 2C, and 2D are schematic diagrams illustrating alternative structures of a sensing region with the aptamer protective material of the exemplary aptamer biosensor as shown in FIG. 2A.

FIGS. 2E and 2F are representative schematics for a linear substrate aptamer construct with aptamer protective material and hypothetical graph of potential vs. electric double layer, respectively in accordance with this disclosure.

FIGS. 2G and 2H are representative schematics for a microporous substrate aptamer construct with aptamer protective material and hypothetical graph of potential vs. electric double layer, respectively.

FIG. 3 is a schematic representation of an exemplary aptamer protective material in accordance with the broadest aspect of the present disclosure.

FIGS. 4A and 4B are a schematic representations of exemplary co-adsorbents in accordance with the broadest aspect of the present disclosure.

FIGS. 5A and 5B are representative graphs of experimental charge vs. frequency data of a control aptamer biosensor verses an exemplary aptamer biosensor with aptamer protective material, respectively, in accordance with this disclosure.

FIGS. 6A and 6B are representative graphs of experimental charge vs. frequency data of protein fouling of a control aptamer biosensor verses an exemplary aptamer biosensor with aptamer protective material, respectively, in accordance with this disclosure.

FIGS. 7A and 7B representative graphs of experimental charge vs. frequency data of protein fouling of a control aptamer biosensor verses an exemplary aptamer biosensor with aptamer protective material, respectively, in accordance with this disclosure.

FIGS. 8A and 8B are representative graphs of experimental current vs. frequency data for an exemplary aminoglycoside aptamer biosensor without and with aptamer protective material, respectively, in accordance with this disclosure.

FIG. 9A is a representative graph of experimental normalized readout percentage vs. time representing drift for a control aptamer biosensor verses an exemplary aptamer biosensor with various aptamer protective materials, exposed to protein, in accordance with this disclosure.

FIG. 9B is a representative graph of experimental normalized readout percentage vs. time representing drift for an exemplary aptamer biosensor with an aptamer protective layer, exposed to bovine serum albumin protein in accordance with this disclosure.

FIG. 9C is a representative graph of experimental normalized readout percentage vs. time representing stability of an exemplary aptamer biosensor with an aptamer protective layer in accordance with this disclosure.

FIGS. 10A and 10B are representative graphs of experimental current vs. frequency data for an exemplary vancomycin aptamer biosensor without and with aptamer protective material, respectively, over time in accordance with this disclosure.

FIG. 11 is a representative graph of experimental sensor response percentage vs. analyte concentration representing an exemplary aptamer biosensor with and without aptamer protective material exposed to various analyte concentrations, in accordance with this disclosure.

FIGS. 12A and 12B are representative graphs of for an exemplary vancomycin aptamer biosensor with different co-adsorbents, respectively, in accordance with this disclosure.

FIGS. 13A and 13B are representative graphs of shelf-life performance uncoated EAB vs an exemplary APL-coated EAB, respectively, after storage for 5 hours in an ambient environment.

FIGS. 13C and 13D are representative graphs of calibration and drift performance of an exemplary vancomycin APL-coated EAB after storage for one month in an ambient environment.

FIGS. 13E and 13F are representative graphs of calibration and drift performance of an exemplary vancomycin APL-coated EAB after storage for two months in an ambient, dark environment.

FIG. 14 is a diagram illustrating certain embodiments of an example continuous analyte monitoring sensor system communicating with at least one display device in accordance with various technologies described in the present disclosure.

### DETAILED DESCRIPTION

Despite significant progress that has been made towards the implementation of AB and EAB devices in vivo, important challenges must be overcome in terms of aptamer stability to facilitate their continuous operation in complex samples such as blood or ISF. There is a need to develop novel EAB interfaces that resist degradation over time as a result of continuous electrochemical interrogation in biological fluids for prolonged periods—a process typically seen as a drop in faradaic and an increase in charging currents over time. This progressive degradation limitsEAB in vivo operational life to 12 hours or less, a period that is much shorter than the elimination half-life of the vast majority of drugs in humans. The present disclosure provides a technical solution to the above problem and facilitates continuous operation of AB and EAB devices in vivo using an aptamer protective material alone or in combination with co-adsorbents.

### Definitions

The term “about” as used herein is a broad term, and is to be given its ordinary and customary meaning to a person of ordinary skill in the art (and is not be limited to a special or customized meaning), and refers without limitation to allowing for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The phrase “substantially free of” as used herein can mean having none or having a trivial amount of, such that the amount of material present does not affect the material properties of the composition including the material, such that about 0 wt. % to about 5 wt. % of the composition is the material, or about 0 wt. % to about 1 wt. %, or about 5 wt. % or less, or less than or equal to about 4.5 wt. %, 4, 3.5, 3, 2.5, 2, 1.5, 1, 0.9, 0.8, 0.7, 0.6, 0.5, 0.4, 0.3, 0.2, 0.1, 0.01, or about 0.001 wt. % or less, or about 0 wt. %.

The term “adhere” and “attach” as used herein are broad terms, and are to be given their ordinary and customary meaning to a person of ordinary skill in the art (and are not be limited to a special or customized meaning), and refer without limitation to hold, bind, or stick, for example, by gluing, bonding, grasping, interpenetrating, or fusing.

The terms “analyte” as used herein is a broad term, and is to be given its ordinary and customary meaning to a person of ordinary skill in the art (and is not to be limited to a special or customized meaning), and refers without limitation to a substance or chemical constituent in a biological fluid (e.g., blood, interstitial fluid, cerebral spinal fluid, lymph fluid, urine, sweat, saliva, etc.) that can be analyzed. Analytes can include naturally occurring substances, artificial substances, drugs, toxins, metabolites, and/or reaction products. Exemplary analytes include, troponin, BNP, insulin, GLP-1, dopamine, serotonin, L-DOPA, vancomycin, aminoglycosides, doxorubicin, cortisol, and Luteinizing Hormone.

The phrases “analyte-measuring device,” “analyte-monitoring device,” “analyte-sensing device,” “continuous analyte sensing device,” “continuous analyte sensor device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte, or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing and/or detecting element.

The term “aptamer” as used herein is a broad term, and is to be given its ordinary and customary meaning to a person of ordinary skill in the art (and is not to be limited to a special or customized meaning), and refers without limitation to an oligonucleotide or a peptide that binds to a biological analyte. Aptamers can be of oligonucleotide or peptide origin. Oligonucleotide aptamers include nucleic acid species that have been engineered through repeated rounds of in vitro selection or equivalently, SELEX (systematic evolution of ligands by exponential enrichment) to bind to biological analytes such as small molecules, proteins, nucleic acids, and even cells, tissues and organisms. Peptide aptamers include polypeptides selected or engineered to bind an analyte. Peptide aptamers can comprise or consist of one or more peptide loops of variable sequence presented in a protein scaffold. Peptide aptamer selection can be made using different systems, including the yeast two-hybrid system, combinatorial peptide libraries constructed by phage display and other surface display technologies such as mRNA display, ribosome display, bacterial display and yeast display, collectively, “biopannings.” Peptides aptamers can be chosen from the MimoDB database. Peptide aptamers can also be isolated from combinatorial libraries created by directed mutation or rounds of variable region mutagenesis and selection. Commercially available aptamers, including aptamers with a transducing element can be purchased, for example, from Biosearch Technologies (Hoddesdon, UK).

The phrase “aptamer conjugate” as used herein, is a broad phrase, and is to be given its ordinary and customary meaning to a person of ordinary skill in the art (and is not to be limited to a special or customized meaning), and refers without limitation to aptamers or bioactive agents covalently linked through a linker to a substrate, co-adsorbate, carrier, or nanocarrier, such as a metal surface, conductive surface, or polymer. The linker can be biologically inactive, as in resisting the separation of the aptamer from the substrate when exposed or presented to a biological environment over a period of time suitable for continuous monitoring, such as with a wearable, or in a subcutaneous or transcutaneous environment, with or without a protective layer. The linker can be biologically active, as in capable of allowing the separation of the bioactive agent (e.g., an anti-inflammatory) from the carrier when exposed or presented to a biological environment, such as a subcutaneous or transcutaneous environment. The phrase “aptamer conjugate” is inclusive of aptamers comprising a linking moiety for coupling or tethering to a substrate, co-adsorbate, carrier, or nanocarrier as well as inclusive of aptamers comprising a linking moiety and a redox moiety coupled thereto.

The phrases “aptamer protective material,” “aptamer protective domain,” “aptamer protective membrane,” “aptamer protective region,” “aptamer protective matrix,” and “aptamer protective layer” as used herein and collectively referred to as “aptamer protective layer 105” or “APL”, are broad phrases, and are to be given their ordinary and customary meaning to a person of ordinary skill in the art (and is not to be limited to a special or customized meaning), and refers without limitation to any substance, domain, membrane, region, polymer, matrix or layer that cooperatively functions with one or more aptamers conjugates configured to transduce a signal corresponding to a concentration of a biological analyte. For example, APL provides one or more of the following attributes: allows an aptamer conjugate to undergo conformational transformations within the APL; allows transport of one or more analytes; provides an electrochemical and/or physiochemical environment about the aptamer for stabilizing the aptamer itself, or its coupling to a substrate, or longevity of a redox moiety coupled to the aptamer; and reduces or eliminates drift of signal over time in vivo.

The phrase and term “bioactive agent” and “bioactive” as used herein is a broad phrase and a broad term, and are to be given their ordinary and customary meaning to a person of ordinary skill in the art (and is not to be limited to a special or customized meaning), and refers without limitation to any substance that has an effect on or elicits a response from living tissue, for example, drugs, biologics, reactive oxygen scavenger (ROS), and metal ions.

The phrases “biointerface membrane,” “biointerface domain,” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, continuous analyte sensing device, continuous analyte sensor device, and/or multi-analyte sensor device responsible for the detection of, or transduction of a signal associated with, a particular analyte or combination of analytes. In examples, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In examples, such biosensors and/or sensors are capable of providing specific quantitative, semi-quantitative, qualitative, semi qualitative analytical signals using a biological recognition element combined with a detecting and/or transducing element.

The term “biostable” as used herein is a broad term, and is to be given its ordinary and customary meaning to a person of ordinary skill in the art (and is not to be limited to a special or customized meaning), and refers without limitation to materials that are relatively resistant to degradation by processes that are encountered in vivo.

The term “co-adsorbate” as used herein is a broad term, and is to be given its ordinary and customary meaning to a person of ordinary skill in the art (and is not to be limited to a special or customized meaning), and refers without limitation to materials that absorb, associate, or couple via covalent, ionic, or molecular interaction to a substrate surface (absorbent). Unless otherwise indicated, a co-adsorbate is at least partially adsorbed onto a surface rather than absorbed into a surface.

The term “comprising” as used herein is synonymous with “including,” “containing,” or “characterized by,” and is inclusive or open-ended and does not exclude additional, unrecited elements or method steps.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrase “continuous analyte sensing” as used herein is a broad phrase, and is to be given its ordinary and customary meaning to a person of ordinary skill in the art (and is not to be limited to a special or customized meaning), and refers without limitation to the period in which monitoring of an analyte concentration is continuously, continually, and/or intermittently (but regularly) performed, for example, from about every 5 seconds or less to about 10 minutes or more. In further examples, continuous monitoring of analyte concentration is performed from about every 10, 15, 20, 25, 30, 35, 40, 45, 50, 55, or 60 second to about 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, continuous monitoring of analyte concentration is performed daily and can be performed for weeks.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another (i.e. “directly coupled”). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of the membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “drift” as used herein is a broad term, and is to be given its ordinary and customary meaning to a person of ordinary skill in the art (and is not to be limited to a special or customized meaning), and refers without limitation to a progressive increase or decrease in signal over time that is unrelated to changes in host systemic analyte concentrations. While not wishing to be bound by theory, it is believed that drift may be the result of a local decrease in analyte transport to the sensor, for example, due to a formation of a foreign body capsule (FBC). It is also believed that an insufficient amount of interstitial fluid surrounding the sensor may result in reduced transport to the sensor. In one example, an increase in local interstitial fluid may slow or reduce drift and thus improve sensor performance. Drift may also be the result of sensor electronics, or algorithmic models used to compensate for noise or other anomalies that can occur with electrical signals in ranges including the milliampere range, microampere range, picoampere range, nanoampere range, and femtoampere range, likewise with faradic, capacitance, and voltage measurements.

The phrases “bioactive releasing membrane” and “drug releasing layer” and “bioactive releasing domain” and “bioactive agent releasing membrane” are used interchangeably herein and are each a broad phrase, and each are to be given its ordinary and customary meaning to a person of ordinary skill in the art (and is not to be limited to a special or customized meaning), and refers without limitation to a permeable or semi-permeable membrane which is permeable to one or more bioactive agents. In examples, the “bioactive releasing membrane” and “drug releasing layer” and “bioactive releasing domain” and “bioactive agent releasing membrane” can be comprised of two or more domains and is typically of a few microns thickness or more. In examples the bioactive releasing membrane and/or bioactive releasing membrane and/or bioactive agent releasing membrane and/or and bioactive agent releasing membrane are substantially the same as the biointerface layer and/or biointerface membrane. In another example, the bioactive releasing membrane and/or bioactive releasing membrane and/or bioactive agent releasing membrane and/or and bioactive agent releasing membrane are distinct from the biointerface layer and/or biointerface membrane.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In another example, electron transfer is provided using a redox moiety associated with an aptamer conjugate, where the redox moiety is capable of undergoing reduction-oxidation (redox) that is related to a reversible binding interaction of the aptamer and an analyte proportional to the analyte concentration.

The term “gain” as used herein is a broad term, and is to be given its ordinary and customary meaning to a person of ordinary skill in the art (and is not to be limited to a special or customized meaning), and refers without limitation to a differential measure between signal OFF state and signal ON state. For example, a typical range of gain is 1-200% of a signal percentage change produced by analyte of certain concentration as compared to zero analyte concentration. Analyte concentration is typically quantified in micromolar (uM), nanomolar (nM), nanograms/milliliter (ng/mL) or picograms/milliliter (pg/mL).

The phrase “hard segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts resistance properties, e.g., resistance to bending or twisting. The term “hard segment” can be further characterized as a crystalline, semi-crystalline, or glassy material with a glass transition temperature determined by dynamic scanning calorimetry (“Tg”) typically above ambient temperature, and is typically made of diisocyanate with or without chain extender.

The term “host” as used herein is a broad term, and is to be given its ordinary and customary meaning to a person of ordinary skill in the art (and is not to be limited to a special or customized meaning), and refers without limitation to mammals, for example humans.

The terms “implanted” or “implantable” as used herein are broad terms, and are to be given their ordinary and customary meaning to a person of ordinary skill in the art (and are not to be limited to a special or customized meaning), and refer without limitation to objects (e.g., sensors) that are inserted subcutaneously (i.e. in the layer of fat between the skin and the muscle) or transcutaneously (i.e. penetrating, entering, or passing through intact skin), which may result in a sensor that has an in vivo portion and an ex vivo portion.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In examples of an electrochemical aptamer sensor, interfering species are compounds with a redox (reduction-oxidation) potential that overlaps with the analyte to be measured or one or more redox moieties associated with one or more aptamers.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term “linker” as used herein is a broad term, and is to be given its ordinary and customary meaning to a person of ordinary skill in the art (and is not to be limited to a special or customized meaning), and without limitation is inclusive of a chemical group or a molecule linking two molecules or moieties, e.g., an aptamer and a substrate, co-adsorbate, carrier, or nanocarrier. In one example, the linker is positioned between, or flanked by, two groups, molecules, or other moieties and connected to each one via a covalent bond, thus connecting the two. In one example, the linker is an oligonucleotide, biotin, maleimide (NHS) esters, polyethylene glycol-NHS esters, or a “click” chemistry component. In one example, thymidine nucleotides of a length from 2-10 with or without a spacer group are used.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to analyte. In examples, the membrane system comprises an immobilized or encapsulated aptamer, which enables transduction to occur between the aptamer and analyte whereby a concentration of analyte can be measured.

The term “micro,” as used herein is a broad term, and is to be given its ordinary and customary meaning to a person of ordinary skill in the art (and is not to be limited to a special or customized meaning), and refers without limitation to a small object or scale of approximately 10⁻⁶ m that is not visible without magnification. The term “micro” is in contrast to the term “macro,” which refers to a large object that may be visible without magnification. Similarly, the term “nano” refers to a small object or scale of approximately 10⁻⁹ m.

The term “noise,” as used herein, is a broad term and is used in its ordinary sense, including, without limitation, a signal detected by the sensor or sensor electronics that is unrelated to analyte concentration and can result in reduced sensor performance. One type of noise has been observed during the few hours (e.g., about 2 to about 24 hours) after sensor insertion. After the first 24 hours, the noise may disappear or diminish, but in some hosts, the noise may last for about three to four days. In some cases, noise can be reduced using predictive modeling, artificial intelligence, and/or algorithmic means. In other cases, noise can be reduced by addressing immune response factors associated with the presence of the implanted sensor, such as using a bioactive releasing membrane with at least one bioactive agent. For example, noise of one or more exemplary biosensors as presently disclosed can be determined and then compared qualitatively or quantitatively. By way of example, by obtaining a raw signal timeseries with a fixed sampling interval (in units of picoampere (pA)), a smoothed version of the raw signal timeseries can be obtained, e.g., by applying a 3rd order lowpass digital Chebyshev Type II filter. Others smoothing algorithms can be used. At each sampling interval, an absolute difference, in units of pA, can be calculated to provide a smoothed timeseries. This smoothed timeseries can be converted into units (the unit of “noise”), using, for example, an analyte sensitivity timeseries, where the analyte sensitivity timeseries is derived by using a mathematical model between the raw signal and reference blood analyte measurements. Optionally, the timeseries can be aggregated as desired, e.g., by hour or day. Comparison of corresponding timeseries between different exemplary biosensors with the presently disclosed bioactive releasing membrane and one or more bioactive agents provides for qualitative or quantitative determination of improvement of noise.

The term “optional” or “optionally” as used herein is a broad term, and is to be given its ordinary and customary meaning to a person of ordinary skill in the art (and is not to be limited to a special or customized meaning), and, without limitation, means that the subsequently described event or circumstance can or cannot occur, and that the description includes instances where the event or circumstance occurs and instances where it does not.

The phrase “polymerization group” used herein is a broad phrase, and is to be given its ordinary and customary meaning to a person of ordinary skill in the art (and is not to be limited to a special or customized meaning), and refers without limitation to a functional group that permits polymerization of the monomer with itself to form a homopolymer or together with different monomers to form a copolymer. Depending on the type of polymerization methods employed, the polymerization group can be selected from alkene, alkyne, epoxide, lactone, amine, hydroxyl, isocyanate, carboxylic acid, anhydride, silane, halide, aldehyde, and carbodiimide.

The term “polyzwitterions” as used herein is a broad term, and is to be given its ordinary and customary meaning to a person of ordinary skill in the art (and is not to be limited to a special or customized meaning), and refers without limitation to polymers where a repeating unit of the polymer chain is a zwitterionic moiety. Polyzwitterions are also known as polybetaines. Since polyzwitterions have both cationic and anionic groups, they are a type of polyampholytic polymer. They are unique, however, because the cationic and anionic groups are both part of the same repeating unit, which means a polyzwitterion has the same number of cationic groups and anionic groups whereas other polyampholytic polymers can have more of one ionic group than the other. Also, polyzwitterions have the cationic group and anionic group as part of a repeating unit. Polyampholytic polymers need not have cationic groups connected to anionic groups; they can be on different repeating units and thus may be distributed apart from one another at random intervals, or one ionic group may outnumber the other.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme layer. If the sensor is deemed to be the point of reference and the enzyme layer is positioned nearer to the sensor than the biointerface layer, then the enzyme layer is more proximal to the sensor than the biointerface layer.

The phrase and term “processor module” and “microprocessor” as used herein are each a broad phrase and term, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer.

The term “semi-continuous” as used herein is a broad term, and is to be given its ordinary and customary meaning to a person of ordinary skill in the art (and is not to be limited to a special or customized meaning), and refers without limitation to a portion, coating, domain, or layer that includes one or more continuous and noncontinuous portions, coatings, domains, or layers. For example, a coating disposed around a sensing region but not about the sensing region is “semi-continuous.”

The phrases “sensing portion,” “sensing membrane,” “sensing region,” “sensing domain,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In examples, the sensing portion, sensing membrane, and/or sensing mechanism generally comprise an electrode configured to provide signals during electrochemically reactions with one or more membranes covering electrochemically reactive surface. In examples, such sensing portions, sensing membranes, and/or sensing mechanisms are capable of providing specific quantitative, semi-quantitative, qualitative, semi qualitative analytical signals using a biological recognition element combined with a detecting and/or transducing element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an aptamer, or RNA or DNA protein, or for example, one or more periplasmic binding protein (PBP) or mutant or fusion protein thereof having one or more analyte binding regions, each region capable of specifically and reversibly binding to at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level in the biological sample.

In examples, the sensing region or sensing portion can comprise at least a portion of a conductive substrate or at least a portion of a conductive surface, for example, a wire or conductive trace or a substantially planar substrate including substantially planar trace(s), and a membrane. In examples, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface.

In one example, multiple working electrodes can be employed. For example, a second working electrode comprising a plurality of different analyte (e.g., analyte 1, analyte2, etc.) aptamer conjugates on the second working electrode to correct for sensor drift and/or interference. Likewise, a second working electrode comprising a non-selective aptamer conjugate to a plurality of different analytes (e.g., analyte 1, analyte2, etc) on the second working electrode can be used to correct for sensor drift and/or interference.

In one example, a combination of at least two sets of identical aptamers, but with one set having a different redox moiety are used to correct for sensor drift and/or interference. In one example, a combination of at least two sets of non-identical aptamer conjugates (e.g., different linker/linker length, coupling chemistry, different selectivity and/or binding affinity) each set having an identical redox moiety are used to correct for sensor drift and/or interference and/or to provide detection within a large physiological analyte concentration range. In one example, a combination of at least two sets of non-identical aptamer conjugates (e.g., different linker/linker length, coupling chemistry, different selectivity and/or binding affinity) each set having a unique redox moiety are used to correct for sensor drift and/or interference and/or to provide detection within a large physiological analyte concentration range. In one example, the same or different aptamer is conjugated to different redox moieties having separated formal potentials so as to reduce or eliminate signals from interfering species.

In another example, the sensing region can comprise one or more periplasmic binding protein (PBP) or mutant or fusion protein thereof having one or more analyte binding regions, each region capable of specifically and reversibly binding to at least one analyte. Mutations of the PBP can contribute to or alter one or more of the binding constants, extended stability of the protein, including thermal stability, to bind the protein to a special encapsulation matrix, membrane or polymer, or to attach a detectable reporter group or “label” to indicate a change in the binding region. Specific examples of changes in the binding region include, but are not limited to, hydrophobic/hydrophilic environmental changes, three-dimensional conformational changes, changes in the orientation of amino acid side chains in the binding region of proteins, and redox states of the binding region. Such changes to the binding region provide for transduction of a detectable signal corresponding to the one or more analytes present in the biological fluid.

In examples, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The sensing region transduces the recognition of analytes into a semi-quantitative or quantitative signal. Thus, “transducing” or “transduction” and their grammatical equivalents as are used herein encompasses optical, electrochemical, acoustical/mechanical, or colorimetrical technologies and methods. Electrochemical properties include current and/or voltage, capacitance, and potential. Optical properties include absorbance, fluorescence/phosphorescence, wavelength shift, phase modulation, bio/chemiluminescence, reflectance, light scattering, and refractive index.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, an amperometric sensor has a sensitivity (or slope) of from about 1 to about 100 picoAmps of current for every 1 mg/dl of analyte.

The phrases and terms “small diameter sensor,” “small structured sensor,” and “micro-sensor” as used herein are broad phrases and terms, and are to be given their ordinary and customary meaning to a person of ordinary skill in the art (and are not to be limited to a special or customized meaning), and refer without limitation to sensing mechanisms that are less than about 2 mm in at least one dimension. In further examples, the sensing mechanisms are less than about 1 mm in at least one dimension. In some examples, the sensing mechanism (sensor) is less than about 0.95, 0.9, 0.85, 0.8, 0.75, 0.7, 0.65, 0.6, 0.5, 0.4, 0.3, 0.2, or 0.1 mm. In some examples, the maximum dimension of an independently measured length, width, diameter, thickness, or circumference of the sensing mechanism does not exceed about 2 mm. In some examples, the sensing mechanism is a needle-type sensor, wherein the diameter is less than about 1 mm, see, for example, U.S. Pat. No. 6,613,379 to Ward et al. and U.S. Pat. No. 7,497,827 to Brister et al., both of which are incorporated herein by reference in their entirety. In some alternate examples, the sensing mechanism includes electrodes deposited on a substantially planar substrate, wherein the thickness of the implantable portion is less than about 1 mm, see, for example U.S. Pat. No. 6,175,752 to Say et al. and U.S. Pat. No. 5,779,665 to Mastrototaro et. al., both of which are incorporated herein by reference in their entirety. Examples of methods of forming the sensors (sensor electrode layouts and membrane) and sensor systems discussed herein may be found in currently pending U.S. Pat. Pub. No. 2019-0307371, which is incorporated by reference in its entirety herein.

The phrase “soft segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts flexibility to the chain. The phrase “soft segment” can be further characterized as an amorphous material with a low Tg, e.g., a Tg not typically higher than ambient temperature or normal mammalian body temperature.

The phrase “solid portions” as used herein is a broad term, and is to be given its ordinary and customary meaning to a person of ordinary skill in the art (and is not to be limited to a special or customized meaning), and refers without limitation to portions of a membrane's material having a mechanical structure that demarcates cavities, voids, or other non-solid portions.

The term and phrases “zwitterion” and “zwitterionic compound” as used herein are each a broad term and phrase, and are to be given their ordinary and customary meaning to a person of ordinary skill in the art (and is not to be limited to a special or customized meaning), and refer without limitation to compounds in which a neutral molecule of the compound has a unit positive and unit negative electrical charge at different locations within the molecule. Such compounds are a type of dipolar compound, and are also sometimes referred to as “inner salts.”

The phrases “zwitterion precursor” or “zwitterionic compound precursor” as used herein are broad phrases, and are to be given their ordinary and customary meaning to a person of ordinary skill in the art (and is not to be limited to a special or customized meaning), and refer without limitation to any compound that is not itself a zwitterion, but can become a zwitterion in a final or transition state through chemical reaction. In some examples described herein, devices comprise zwitterion precursors that can be converted to zwitterions prior to in vivo implantation of the device. Alternately, in some examples described herein, devices comprise zwitterion precursors that can be converted to zwitterions by some chemical reaction that occurs after in vivo implantation of the device. Such reactions are known to a person of ordinary skill in the art and include ring opening reaction, addition reaction such as Michael addition. This method is especially useful when the polymerization of betaine containing monomer is difficult due to technical challenges such as solubility of betaine monomer to achieve desired physical properties such as molecular weight and mechanical strength. Post-polymerization modification or conversion of betaine precursor can be a practical way to achieve desired polymer structure and composition. Examples of such as precursors include tertiary amines, quaternary amines, pyridines, and others detailed herein.

The phrases “zwitterion derivative” or “zwitterionic compound derivative” as used herein are broad phrases, and are to be given their ordinary and customary meaning to a person of ordinary skill in the art (and is not to be limited to a special or customized meaning), and refer without limitation to any compound that is not itself a zwitterion, but rather is the product of a chemical reaction where a zwitterion is converted to a non-zwitterion. Such reactions can be reversible, such that under certain conditions zwitterion derivatives can act as zwitterion precursors. For example, hydrolysable betaine esters formed from zwitterionic betaines are cationic zwitterion derivatives that under the appropriate conditions are capable of undergoing hydrolysis to revert to zwitterionic betaines.

The phrases “zwitterionic repeating group” as used herein is a broad phrase, and is to be given their ordinary and customary meaning to a person of ordinary skill in the art (and is not to be limited to a special or customized meaning), and refer without limitation to, independently, two or more zwitterionic compounds, zwitterion derivatives or zwitterionic compound derivatives in the same compound or polymer.

Aptamer-based biosensors (AB's) and electrochemical aptamer-based biosensors (EAB's) are analytical platforms that can provide for continuous monitoring of specific molecular analytes, in vivo. EAB sensors typically present an architecture consisting of a self-assembled monolayer (SAM) of analyte-binding, alkanethiol-functionalized nucleic-acid aptamers or other bioreceptor comprising a redox moiety sensitive as signal transducing element to correlate analyte-binding events with measurable electrical energy changes, a SAM of electrode-blocking co-adsorbents of alkanethiols to prevent undesired electrochemical reactions and confer biocompatibility to the electrode surface.

The poor stability observed when the above AB or EAB's are placed in physiological relevant environments is at least attributed to desorption of the aptamer monolayer from a substrate surface or desorption of the underlying SAM monolayer used to immobilize the aptamer or the electrode-blocking SAM, as well as bioelectronic interface degradation (e.g., fouling, drift, etc.) upon continuous electrochemical interrogation-a process typically seen as a drop in faradaic and an increase in charging currents over time. As discussed in greater detail below, such performance deficits can be addressed with the presently disclosed aptamer protective layer (APL).

In one example, the present disclosure provides for AB or EAB's in an architecture consisting of a self-assembled monolayer (SAM) of analyte-binding, alkanethiol-or carboxyl-functionalized nucleic-acid aptamers or other bioreceptor comprising a signal transducing element to correlate analyte-binding events with a measurable signal from the transducing element, SAM of electrode-blocking co-adsorbents of alkanethiols and/or functionalized alkanethiols and an aptamer protective layer (APL) adjacent or directly adjacent the above architecture to independently or collectively prevent undesired desorption, undesired reactions, reduce biofouling,/confer biocompatibility, aptamer stability, and device longevity.

In one example, the aptamer conjugate and APL are temperature controlled during use, for example, a wearable sensor is thermally insulated, and/or configured with a mini-Peltier cooler and/or heat exchange devices, e.g., fins, or combinations of the above. In another example, the aptamer is prepared (e.g., Systematic Evolution of Ligands by Exponential Enrichment (SELEX)) under conditions closely matched to the in vivo thermodynamic environment of the sensor, so that high affinity and/or thermal stability is provided or improved.

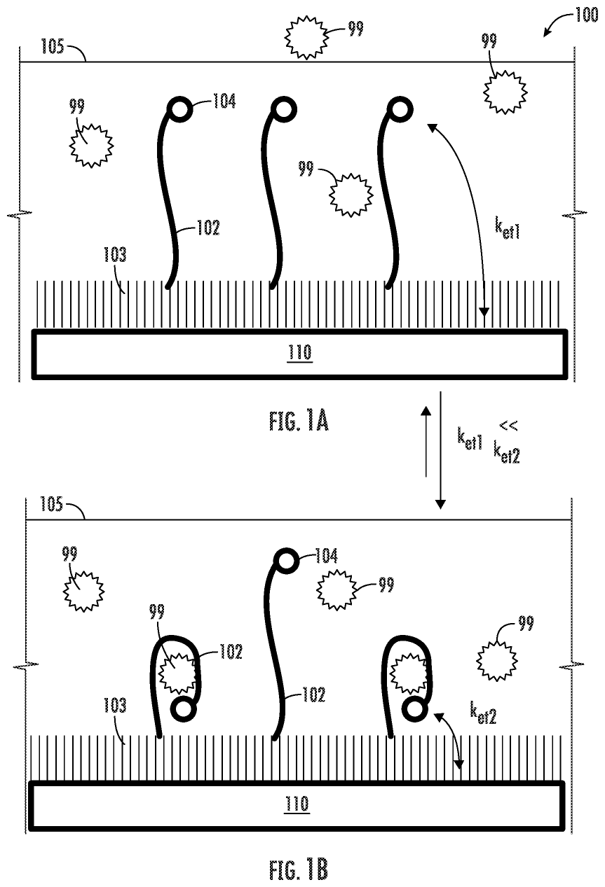

With reference to FIGS. 1A and 1B exemplary aptamer-based analyte monitoring sensor 100 configured for in vivo measurement of at least one analyte 99, is presented with schematic diagrams illustrating an aptamer protective material 105. Aptamer 102 with signal transducing element 104 is shown associated with optional monolayer 103 adjacent substrate 110. Monolayer 103 can be covalently or non-covalently coupled to substrate 110. In one example, aptamer 102 undergoes a reversible conformational change upon interaction with analyte 99, e.g., an analyte, metabolite, drug, etc., causing signal transducing element 104 to present in closer proximity to substrate 110 so as to provide a signal corresponding to the analyte 99 concentration or presence.

In one example, the signal transducing element 104 is a reversible redox moiety and the substrate 110 is electrically conductive and the reversible binding of aptamer 102 (and its subsequent reversible conformational change) upon interaction with analyte 99 causes a change in proximity of all or part of the signal transducing element 104 with that of the conductive substrate 110 such that the signal transducing element 104 is capable of undergoing detectable reversible reduction-oxidation reaction(s) via electron transfer with the conductive substrate 110 upon reversible binding of analyte 99 with aptamer 102. The detectable reversible reduction-oxidation reaction(s) via electron transfer with the conductive substrate 110 provide for correlation with analyte 99 concentration as further discussed below.

In another example, the reversible binding of aptamer 102 (and its subsequent reversible conformational change) upon interaction with analyte 99 can cause all or part of signal transducing element 104 to present in or to a different local environment, for example, from a hydrophobic local environment to a hydrophilic local environment (or visa-versa) so as to provide a detectable signal corresponding to analyte 99 concentration or presence.

In one example, the signal transducing element 104 is an environmentally sensitive fluorescent dye or phosphorescence dye that upon exposure to electromagnetic radiation, e.g., light, is capable of undergoing a detectable change in emission wavelength or frequency and/or emission relaxation or emission decay rate, for example, upon reversible binding with analyte 99 and reversible conformation change from a hydrophobic local environment to a hydrophilic local environment (or visa-versa). The detectable change in emission wavelength or frequency and/or emission relaxation or emission decay rate so as to provide for correlation with analyte 99 concentration.

Signal transducing element 104 can be covalently or non-covalently coupled to aptamer 102, where the covalently or non-covalently coupling is such that it is sufficient for continuous signal transduction of signal over a time period commensurate with a transdermal, intradermal, subcutaneous, ocular, or skin based continuous analyte sensing devices. In one example, continuous signal transduction of signal over a time period of at least 12 hours, at least 24 hours, at least 36 hours, at least 48 hours, at least one week, at least 2 weeks, at least 3 weeks, using the presently disclosed aptamer protective material 105 is envisaged.

In one example, the signal transducing element 104 and aptamer 102 are conjugated or form a conjugate. In one example, the signal transducing element 104 and aptamer 102 conjugate are associated with monolayer 103. In one example, the signal transducing element 104 and aptamer 102 conjugate are covalently or non-covalently coupled to monolayer 103. In one example, the signal transducing element 104 and aptamer 102 conjugate are covalently or non-covalently coupled to substrate 110.

By way of example, any hereafter reference to a redox moiety as the signal transducing element 104 is for brevity and is not to limit the scope of signal transducing element 104. Thus, “redox moiety 104” and “signal transducing element 104” are hereinafter used interchangeably.

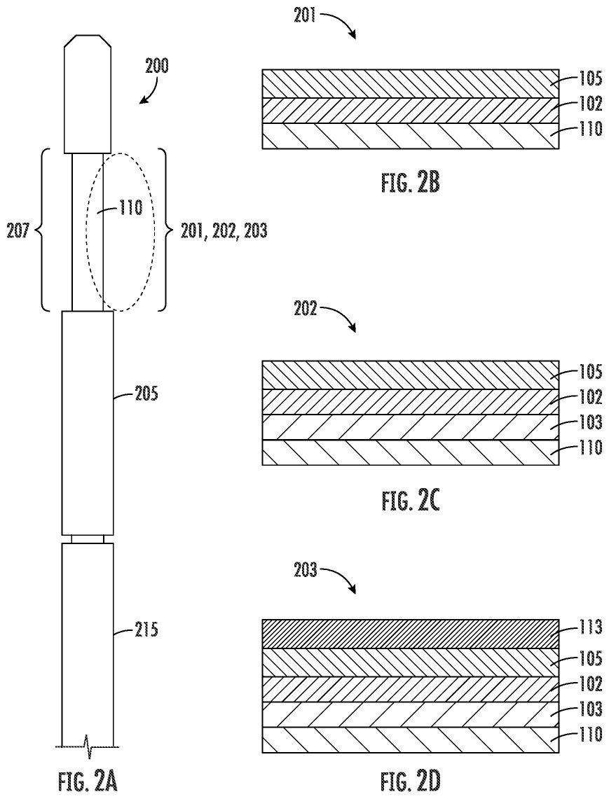

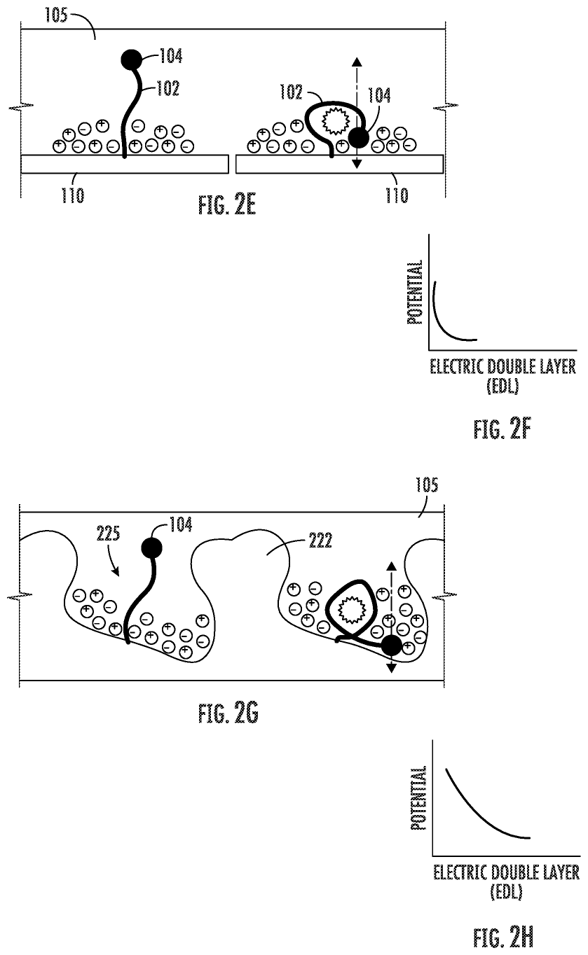

FIG. 2A is a schematic diagram illustrating an exemplary aptamer biosensor 200 construct configured for continuous in vivo use in a subject. Thus biosensor 200 is shown as an elongated member having a sensing region 207, for example, created from a window in an electrically insulated coating 205 about a conductive wire. Alternatively, a window can be prepared in a jacket of an optical fiber for use with an optically based AB device. Additional electrodes 215 (reference electrode and/or counter electrode) can be used or provided separately, for example, as an adjacent co-axial elongated member. As shown in enlarged section views FIGS. 2B-2D, alternative structures 201, 202, and 203 of sensing region 207 are shown with substrate 110 surface, for example, structure 201 has substrate 110 surface with adjacent aptamer 102 and aptamer protective material 105. Structure 202 has substrate 110 surface with adjacent co-adsorbate 103, aptamer 102 and aptamer protective material 105. Structure 203 has substrate 110 surface adjacent co-adsorbate 103, aptamer 102, aptamer protective material 105 and drug-releasing membrane 113 most distal from substrate 110. Other configurations of the co-adsorbate 103, aptamer 102, aptamer protective material 105 and drug-releasing membrane 113 can be employed.

### Substrate/Electrodes

In examples, substrate 110 surface accepts the AB or EAB for use in a continuous sensing device. In one example, substrate 110 is or comprises a conductive material. In one example, substrate 110 is an electrode that may be a wire, a planar structure or a substantially planar structure. In one example, substrate 110 can be configured to provide, independently, one or more of a working electrode, a reference electrode and optionally a counter electrode. In one example, the one or more of the working electrode, the reference electrode and optionally the counter electrode are arranged in a linear or substantially linear configuration. In one example, the reference electrode comprises silver (Ag) and/or silver chloride (AgCl). In one example, the reference electrode comprises silver (Ag) and/or silver chloride (AgCl) encapsulated or otherwise covered with a protective layer. In one example, the reference electrode with a protective layer is configured to reduce or eliminate diffusion of AgCl⁺, Ag, AgCl⁻², ions or particles from the reference electrode and/or reduce or eliminate interaction of the reference electrode or AgCl+ ion with the aptamer and/or a thiol-containing co-adsorbate or a thiol-containing aptamer protective layer. In one example the protective layer of the silver reference electrode is configured to inhibit or reduce transport of AgCl+ ion while allowing transport of chloride ion. Examples of protective layers suitable for the silver reference electrode include, but are not limited to amphiphilic polyurethane or polyurethane urea, Teflon, microporous Teflon, ion selective membranes, semi-permeable membranes, PVC, and plasticized PVC.

In one example, substrate 110 comprises a wire formed from or coated with a conductive material, such as platinum, platinum-iridium, palladium, graphite, gold, carbon, graphene, graphene oxide, conductive polymer, alloys, or the like.

In one example, at least a portion of substrate 110 comprises pores having average pore diameters with nanometer and/or micrometer dimensions. Such pore diameters dimensions in the aforementioned substrates can be formed with etching or plasma techniques, for example. Substrates with such nanometer and/or micrometer dimensions can be used in combination with the presently disclosed APL's.

For example, the structural nature of the substrate can be determinative in the performance of an EAB, as shown in FIGS. 2E and 2F, a planar substrate 110 with aptamer 102 and coupled redox moiety 104 present the shown potential vs electric double layer (EDL) relationship, a region contained within a Debye volume. In contrast, FIGS. 2G shows the same with aptamer 102 and coupled redox moiety 104 construct with at least a portion of a nanometer and/or micrometer dimensioned pores 225 in substrate 222 surface, where as shown in FIG. 2H, the potential vs electric double layer (EDL) relationship presents a smaller relative negative slope compared to the linear substrate. When used in combination with the presently disclosed APL, substrate 222 surfaces can provide increases in signal and limit of detection for continuous EAB devices. In addition, the above construct can provide redox moiety intercalation among closely spaced aptamers, which can offer two sites to absorb two analytes through (pi-pi) π-π interaction of select analytes-redox moieties (e.g., methylene blue and dopamine), rather than just one analyte absorption on an aptamer alone, thereby doubling the detection sensitivity.

In one example, substrate 110 surface comprises a conductive surface and at least a portion of substrate 110 surface is configured to covalently couple, tether, or associate the aptamer conjugate to couple or tether to the conductive surface. In one example, the aptamer conjugate is coupled or tethered to the conductive surface by providing suitable coupling or tethering functionality on one or both of the conductive surface and aptamer and coupling or tethering the aptamer conjugate to the conductive surface using one or more coupling chemistries, for example, chemistry click using N-(3-dimethylaminopropyl)-N′-ethylcarbodiimide/N-hydroxysulfosuccinimide (EDC/NHS) chemistry, silane-based coupling, using diazonium salt/thiolene click chemistry, phosphonate coupling, (strept) avidin-biotin coupling, silane coupling, pi-pi stacking, bicyclo[6.1.0]nonyne (BCN)-azide coupling, biotinylation, Cu(I)-catalyzed azide-alkyne click chemistry reaction (CuAAC), ligation of tetrazine and alkene (e.g., using trans-cyclooctene), and biocompatible strain-promoted azide-alkyne click chemistry (SPAAC) reagents, dibenzocyclooctyne (DBCO)-azide or DBCO-NHS regents, DBCO-PEG-amine coupling regents, DBCO-PEG-maleimide coupling regents, DBCO-PEG-alcohol reagents, amine-reactive trans-cycloctene (TCO) reagents for tetrazine coupling, e.g., TCO-NHS esters, carboxyl/carbonyl reactive TCO regents (e.g., TCO amine or amine salts), TCO-PEG-DBCO reagents, and the like. In other examples, polyethylene glycol (PEG) linkers are used.

In one example, at least a portion of substrate 110 surface comprises carbon, graphene, graphene oxide, or carbon ink. In one example at least a portion of substrate surface is comprised of nanomaterial. In one example, at least a portion of substrate 110 surface comprises carbon, graphene, or graphene oxide nanomaterial. In one example, the use of carbon, graphene, or graphene oxide nanomaterials improve aptamer loading onto substrate 110 surface to optimize aptamer 102 load and binding stability, among other things. In one example, the aptamer conjugate is configured for electrografting to a carbon-based electrode using diazonium salt/thiolene click chemistry, phosphonate coupling, (strept) avidin-biotin coupling, silane coupling, pi-pi stacking, bicyclo[6.1.0]nonyne (BCN)-azide coupling, biotinylation, Cu(I)-catalyzed azide-alkyne click chemistry reaction (CuAAC), ligation of tetrazine and alkene (e.g., using trans-cyclooctene), and biocompatible strain-promoted azide-alkyne click chemistry (SPAAC) reagents, dibenzocyclooctyne (DBCO)-azide or DBCO-NHS regents, DBCO-PEG-amine coupling regents, DBCO-PEG-maleimide coupling regents, DBCO-PEG-alcohol reagents, amine-reactive trans-cycloctene (TCO) reagents for tetrazine coupling, e.g., TCO-NHS esters, carboxyl/carbonyl reactive TCO regents (e.g., TCO amine or amine salts), TCO-PEG-DBCO reagents, and the like. In other examples, polyethylene glycol (PEG) linkers for the aptamer conjugate are used to increase resistance to nucleases, or lipid conjugation to the aptamer is used, or alternative nucleic acids for aptamer construction (e.g., L-DNA, or L-RNA with increased —OH activity for coupling/tethering are used, as well as the use of peptide nucleic acid (PNA) for aptamer construction and combinations of the above.

In one example, substrate 110 surface comprises gold and at least a portion of substrate 110 surface is configured to covalently couple, tether, or associate an alkyl thiol or mercaptothiol. In another example, at least a portion of substrate 110 surface is configured to covalently couple linear or branched aliphatic amine, substituted or unsubstituted benzyl amine, or substituted or unsubstituted phenylamine or covalently couple linear or branched amino alkanoic acid, substituted or unsubstituted amino benzylic acid, or substituted or unsubstituted amino phenyl carboxylic acid. In one example, at least a portion of substrate 110 surface is chemically modified with streptavidin, avidin, gold, biotin, or polymers such as dextrine and chitosan. In one example, a substrate comprising a gold surface is modified or treated with graphene oxide and/or zinc sulfide (ZnS2) to improve aptamer coupling or tethering to the substrate.

In one example, at least a portion of carbon, graphene, or graphene oxide nanomaterial substrate 110 surface comprises covalently coupled linear or branched aliphatic amine, substituted or unsubstituted benzyl amine, or substituted or unsubstituted phenylamine or covalently couple linear or branched amino alkanoic acid, substituted or unsubstituted amino benzylic acid, or substituted or unsubstituted amino phenyl carboxylic acid. In one example, the linear or branched aliphatic amine, substituted or unsubstituted benzyl amine, or substituted or unsubstituted phenylamine or covalently couple linear or branched amino alkanoic acid, substituted or unsubstituted amino benzylic acid, or substituted or unsubstituted amino phenyl carboxylic acid is also covalently coupled to aptamer 102 or aptamer-redox moiety 104 conjugates. For example, substrate 110 surface is modified with a carboxylated material to enable covalent immobilization with exposed COOH— groups of an amine-modified aptamer via EDC/NHS chemistry.

Among exemplary nanomaterials, graphene oxide (GO) shows significant advantages for use in EAB devices due to its large surface area with multiple exposed carboxylic groups (COOH) and alcohol groups (COH) which can be used as anchor points to immobilize aptamer conjugate probes using a variety of different types of coupling chemistries. GO offers great versatility of functionalization and has demonstrated beneficial orientation effects in aptamer immobilization. Thus, in one example a partially or fully implantable-type sensor with a GO-functionalized substrate 110 surface (as a model carboxylated-surface) to serve as working electrode for the covalent immobilization of amine-functionalized aptamers. To covalently immobilize the aptamer conjugates to the GO surface, for example, activation of the GO-carboxyl (‘COOH’) moieties can be performed via N-(3-dimethylaminopropyl)-N′-ethylcarbodiimide/Nhydroxysulfosuccinimide (EDC/NHS) chemistry, then forming the corresponding amide bond with the amine group present in the aptamer sequence. This immobilization strategy is envisaged to provide higher stability of the immobilized aptamer monolayer on the GO electrode surface, thus offering extended sensor lifetime and representing an alternative to thiol-based aptamer immobilization strategies. In a similar manner, activation of the GO-‘COH’ moieties with trialkoxysilane modified aptamer conjugate can be performed. In one example, the aptamer conjugate can be electrochemically grafted to the GO electrode surface.

In one example, substrate 110 surface is a carboxyl-functionalized substrate 110 surface, a thiol functionalized substrate 110 surface, or a combination of a carboxyl-functionalized substrate 110 surface and a thiol functionalized substrate 110 surface.

In one example, substrate 110 surface is substantially a carboxyl-functionalized substrate 110 surface. In one example, substrate 110 surface is essentially a carboxyl-functionalized substrate 110 surface substantially without thiol functionalization.

In one example, substrate 110 surface is substantially a GO-functionalized substrate 110 surface. In one example, substrate 110 surface is essentially a GO-functionalized substrate 110 surface substantially without thiol functionalization.

### Aptamer/Aptamer-Signal Transducing Element Conjugates

In one example, the one or more aptamer conjugates 102 of the presently disclosed AB or EAB comprises RNA or DNA nucleotide sequences. In one example, the one or more aptamer conjugates 102 comprises at least one of: 2′-O-methyl modification of a nucleotide; disulfide bridges; a 3′ cap with an inverted 2-deoxy thymidine; a 3′-3′-thymidine linkage at 3′ terminus; a 2′-F modification; and a double stranded section. In one example, the one or more aptamer conjugates 102 comprises RNA or DNA sequences with a first linker moiety on a 5′ end and the reversible redox moiety at a 3′ end. In one example, the one or more aptamer conjugates 102 comprises RNA or DNA sequences with a first linker moiety on a 3′ end and the reversible redox moiety at a 5′ end. In one example, a redox moiety, e.g., methylene blue, is be attached to an oligo portion of the aptamer, either internal to the sequence linked though thymidine base, at the 5′ end of the sequence, or at the 3′ end though a modified thymidine or 5-7-carbon spacer.

In one example, the first linker moiety on the 5′ end of aptamer 102 comprises an amino group, carboxyl group, or trialkoxylsilane group. In one example, the first linker moiety of aptamer 102 is physically or chemically coupled to the substrate at the 5′ end. In one example, the first linker moiety of aptamer 102 is physically or chemically coupled to the co-adsorbate at the 5′ end. Alternatively, the first linker moiety on the 3′ end of aptamer 102 comprises an amino group, carboxyl group, or trialkoxylsilane group and the first linker moiety of aptamer 102 is physically or chemically coupled to the substrate at the 3′ end. In one example, the first linker moiety of aptamer 102 is physically or chemically coupled to the co-adsorbate at the 3′ end.

In one example, the one or more aptamer conjugates 102 is a neurotransmitter binding aptamer. In one example, the one or more aptamer conjugates 102 is a dopamine or glutamate binding aptamer. In one example, the one or more aptamer conjugates 102 is a carbohydrate, triglyceride or fatty acid binding aptamer. In one example, the one or more aptamer conjugates 102 is a glucose, glycerol, or beta-hydroxybutyrate binding aptamer. In one example, the one or more aptamer conjugates 102 is a glycopeptide antibiotic binding aptamer. In one example, the one or more aptamer conjugates 102 is a vancomycin binding aptamer. Combinations of different aptamer conjugates 102 on the same or different WE surfaces can be employed for providing a multi-analyte monitoring EAB device.

In one example, the one or more aptamer conjugates is physically or chemically coupled to a self-assembled monolayer (SAM). In one example, the one or more aptamer conjugates is physically or chemically coupled to a mono-functional or multi-functional alkanethiol or mercaptoalkanol. In one example, the one or more aptamer conjugates is physically or chemically coupled to an alkylthiol betaine. In one example, the one or more aptamer conjugates is physically or chemically coupled to an aliphatic amine.

### Aptamer Protective Layer (APL)

It has been observed that specific attributes of the APL effect the suitability of continuous operation of an AB or EAB in vivo. For example, a continuous AB or EAB having an APL with sufficient free volume for aptamer conformational changes, favorable ionic characteristics, sufficient porosity to analyte and blocking of proteins, peptides, macrophages and other immune response biologics positively effects the EAB performance. One or more of the above characteristics further provides for, directly or indirectly, stability of the aptamer coupling or tethering (reduced desorption from substrate or SAM), reduced signal or sensitivity drift over in vivo time, and extended in vivo performance compared to an AB or EAB without an APL.

In one example, the APL is a coating, matrix, membrane, domain, or layer. In another example, the APL is a coating, matrix, membrane, domain, or layer of a polymeric material. The polymeric material that forms a basis of an APL can include one or more polymers, oligomers, layers of coatings, membranes, or matrixes. In one example, the APL provides sufficient permeability to allow relevant analyte compounds to pass through it, for example, to allow the analyte to pass through the membrane from the sample under examination in order to reach the aptamer and allow for transduction of a signal corresponding to the analyte concentration in the sample.

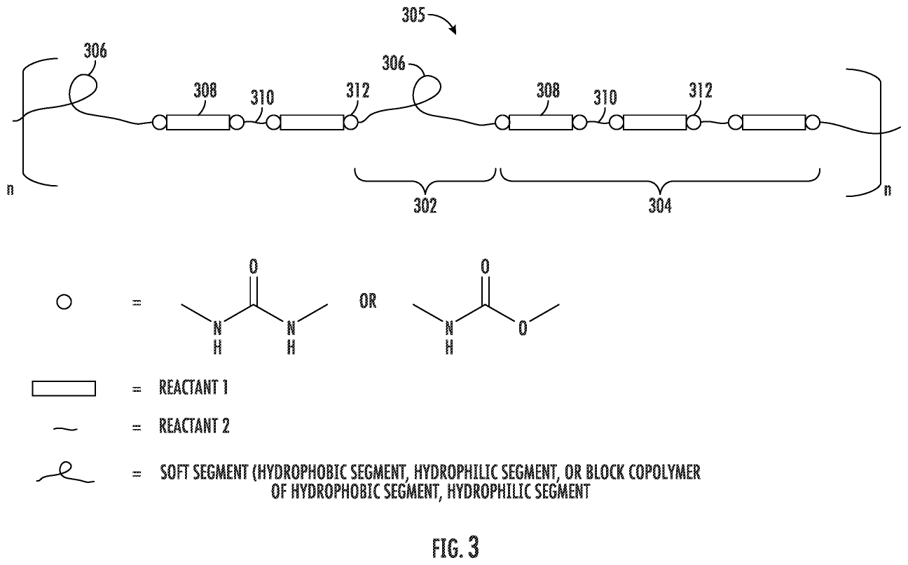

FIG. 3 is a schematic representation of an exemplary APL in accordance with the broadest aspect of the present disclosure., Thus, in one example, APL 305 comprises at least one polymer segment 302, 304. In one example, the APL comprises at least one polymer segment selected from the group consisting of polyurethane, polyurea, poly (urethane urea), epoxide, polyolefin, polysiloxane, polyamide, polystyrene, polyacrylate, polyether, polyvinylpyridine, polyvinylpyrrolidone, polyester, polycarbonate, and copolymers thereof.

The hydrophilicity of APL 305 can be adjusted by the selection of soft segment used and soft segment ratio during conventional PU or PUU synthesis. For example, soft segment components are shown in FIG. 3, (hydrophilic and hydrophobic polyols). Hydrophobic soft segment can be PDMS, polycarbonates, polyester, polyether or polymers with hydrophobic functional groups such as fluorine or silicone. The hydrophobic segment can be provided in the aforementioned APL to be between 1-50 wt. %, 2-50 wt. %, 5-50 wt. %, 10-50 wt. %, 15-50 wt. %, 20-50 wt. %, 25-50 wt. %, 30-50 wt. %, 10-20 wt. %, 15-25 wt. %.

Hydrophilic soft segments can be polyethylene glycols, oligo polyether, polyoxazoline (POX), polypeptide, or zwitterionic polymer. By tuning the chemical composition and/or molecular weight or distribution of soft and hard segment in the PU or PUU, and/or addition or exclusion of functional groups, desirable APL properties and functionality can be achieved, e.g., surface charge/density, antifouling properties against protein such as serum albumin (SA). The hydrophilic segment can be provided in the aforementioned APL to be between 1-50 wt. %, 2-50wt. %, 5-50 wt. %, 10-50 wt. %, 15-50 wt. %, 20-50 wt. %, 25-50 wt. %, 30-50 wt. %, 10-20 wt. %, 15-25 wt. %.

In one example, the APL comprises a segmented multiblock polymer. With reference again to FIG. 3, for example, the segmented multiblock polymer comprises a soft segment 306 and a hard segment (one or more of 308, 310, 312). In one example, the soft segment is hydrophobic or hydrophilic. In one example, the soft segment is hydrophobic and hydrophilic. In one example, the soft segment comprises hydrophobic polyol and hydrophilic polyol. In one example, the APL comprises a segmented multiblock polyurethane polymer. In one example, the APL comprises a segmented multiblock polyurethane, polyurethane-urea, or polyether-urethane, or polyether-urethane-urea polymer, copolymer or blends thereof. In one example, the hard segment comprises urethane groups, urea groups, or combinations thereof.

In one example, the soft segment is one or more segments comprising polydimethylsiloxane, polycarbonate, polyester, polyether, and blends or copolymers thereof. In one example, the soft segment is one or more segments comprising polyethylene glycol, oligo polyether, polyoxazoline (POX), polypeptide, polyvinylpyrrolidone, polyvinylpyridine, a polymer with repeating zwitterionic groups on its backbone and/or termini, herein referred to as “zwitterionic repeating group polymer,” and blends or copolymers thereof. In one example, end group functionalized-polyurethane (EGFPU) or polyurethane urea (EGFPUU) polymers can be used. EGFU/EGFPUU's can be synthesized using reactive functional monomers/oligomers end capping the polyurethane reaction intermediates to form polyurethane with functional groups on one chain end or both chain ends. The functional groups could be further deprotected to form reactive thiol groups connect to substrate 110 surface, e.g., gold.

Polyurethane, polyurethane-urea polymer can be produced by the condensation reaction of a diisocyanate and a difunctional hydroxyl-containing material or difunctional amine containing material. A polyurethane-urea is a polymer produced by the condensation reaction of a diisocyanate and a difunctional amine-containing material. In some examples, diisocyanates include aliphatic diisocyanates containing from about 4 to about 8 methylene units. Di isocyanates containing cycloaliphatic moieties can also be useful in the preparation of the polymer and copolymer components of the membranes of the present disclosure.

In one example, end group functionalized-polyurethane (EGFPU) or polyurethane urea (EGFPUU) polymers can be used, for example, as disclosed in co-assigned U.S. Pat. No. 10,413,227B2. EGFU/EGFPUU's can be synthesized using reactive functional monomers/oligomers end capping the polyurethane reaction intermediates to form polyurethane with functional groups on one chain end or both chain ends. The functional groups could be further deprotected to form reactive thiol groups connect to substrate 110 surface, e.g., gold.

For example, an exemplary APL hydrophobic-hydrophilic segmented copolymer component is a polyurethane polymer that includes about 20% hydrophilic polyethylene oxide. The polyethylene oxide portions of the copolymer are thermodynamically driven to separate from the hydrophobic portions of the copolymer and the hydrophobic polymer component. In one example, it has been observed that about 20% polyethylene oxide-based soft segment portion of the copolymer used to form the APL affects the water pick-up and subsequent analyte permeability of the APL membrane. In one example, the aforementioned exemplary APL's are prepared as water-borne dispersions for use with the aforementioned aptamer-signal transducing element conjugates. For example, a hydrophilic aliphatic polyurethane, betaine functionalized, can be prepared as an aqueous dispersion that can be combined with an aqueous solution of the aforementioned aptamer-signal transducing element conjugates.

Incorporation of zwitterionic repeating units into the aforementioned polyurethane, polyurethane-urea polymer a polymer can be achieved by using zwitterionic monomers that have diols or diamines, or can be attached to diols or diamines. Examples of such zwitterionic monomers include:

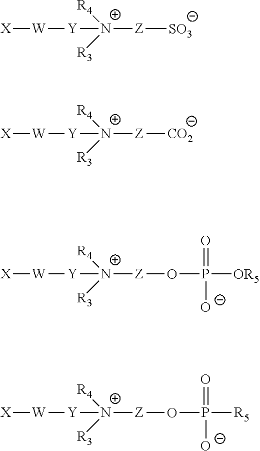

where X is one or two of —OH, —NHR1, —NH2, or —SH; where W, Y, and Z are, independently, branched or straight chain alkyl, heteroalkyl, cycloalkyl, cycloheteroalkyl, aryl, or heteroaryl, any of which can be optionally substituted with O, OH, halogen, amido, or alkoxyl; R1 is H, branched or unbranched acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl; and R3, R4, and R5, are independently chosen from acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl.

These compounds can be reacted with a diisocyanate to form a polyurethane or polyurea comprising zwitterionic repeating units. Alternatively, the carboxylates, sulfonates, phosphinates, or phophonates moieties of the precursor zwitterionic repeating units can be protected and then the protection group can be removed after polymerization. In another alternative, the amine can be a tertiary amine, which is then quaternized by alkylation after polymerization. Further examples of PU and PUU polymers with zwitterionic repeating units can be found in co-assigned U.S. Pub. Appl. No. 20170188923, U.S. Pat. Nos. 11,112,377, and 11,179,079, the disclosure pertaining to such polymers and their synthesis being incorporated by reference herein.

In one example, the APL is comprised of non-polyurethane polymer. Examples of materials which can be used to make non-polyurethane type APL's include vinyl polymers, polyethers, polyesters, polyamides, polysilicones poly (dialkylsiloxanes), poly (alkylarylsiloxanes), poly (diarylsiloxanes), polycarbosiloxanes, polycarbonate, Nafion (sulfonated tetrafluoroethylene) natural polymers such as cellulosic and protein-based materials, and mixtures, copolymers, or combinations thereof with or without the aforementioned polyurethane, or polyether-urethane-urea polymers.

APL's disclosed herein can be formulated into mixtures that can be drawn into a film or applied to a surface using any method known in the art (e.g., spraying, painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., 50-150° C.). Other suitable curing methods can include ultraviolet or gamma radiation, for example.

In one example, the aptamer protective layer is at least partially cross-linked using an amount of cross-linking agent sufficient to crosslink the APL without deactivation of the aptamer or substantial reduction in the ability of the aptamer present therein to undergo conformation change sufficient to provide for signal transduction. In one example, the aptamer protective layer is completely cross-linked using an amount of cross-linking agent sufficient to crosslink the APL without substantial reduction in the aptamer signal transduction.

Suitable cross-linking agents include isocyanate, carbodiimide, gluteraldehyde or other aldehydes, aziridine, silane, epoxy, acrylates, free-radical based agents, ethylene glycol diglycidyl ether (EGDE), poly(ethylene glycol) diglycidyl ether (PEGDE), dicumyl peroxide (DCP), PVP-PEGDE or PVP-PEG. In one embodiment, from about 0.1% to about 15% w/w of cross-linking agent is added relative to the total dry weights of cross-linking agent and polymers added when blending the ingredients (in one example, about 1% to about 10%). During the curing process, substantially all of the cross-linking agent is believed to react, leaving substantially no detectable unreacted cross-linking agent in the final layer.

In one example, the APL is a conductive polymer. In one example, the APL is a functionalized polymer. The APL can be functionalized, for example, with between 1-50 wt. %, 2-50 wt. %, 5-50 wt. %, 10-50 wt. %, 15-50 wt. %, 20-50 wt. %, 25-50 wt. %, 30-50 wt. %, 10-20 wt. %, 15-25 wt. %, of functional moiety. The functionalized polymer can be configured to couple with the substrate, or a SAM, or another layer, membrane, matrix, region, or polymer.

In one example, the functionalized polymer comprises alkanethiol groups. In one example, the alkanethiol groups are present at the end of the functionalized polymer chain or the alkanethiol groups are present along the backbone of the functionalized polymer chain.

In one example, the functionalized polymer comprises mercaptoalkanol groups. In one example, the mercaptoalkanol groups are present at the end of the functionalized polymer chain or are present along the backbone of the functionalized polymer chain.

In one example, the APL comprises a zwitterionic group compound or a zwitterionic repeating group compound. In one example the functionalized polymer is prepared with at least one of a polymerizable zwitterionic monomer structure as follows:

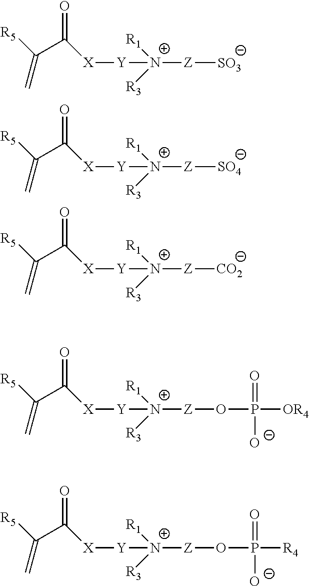

where X is O, NH, or NR₄, Y and Z are, independently, branched or straight chain alkyl, heteroalkyl, cycloalkyl, cycloheteroalkyl, aryl, or heteroaryl, and of which can be optionally substituted with OH, halogen, or alkoxyl; R₁, R₃, R₄, and R₅ are independently H, alkyl, heteroalkyl, cycloalkyl, cycloheteroalkyl, aryl, or heteroaryl.

For example, the APL can comprise, alone or in combination with other polymer structure/backbone, zwitterionic monomers including N-(2-methacryloyloxy) ethyl-N,N-dimethylammonio propanesulfonate, N-(3-methacryloylimino)propyl-N,N-dimethylammonio propanesulfonate, 2-(methacryloyloxy)ethylphosphatidylcholine, and 3-(2′-vinyl-pyridinio)propanesulfonate.

In one example, the presently disclosed APL provides an amount of a zwitterionic repeating group compound capable of modulating or maintaining an ionic strength or pH about the aptamer and/or transducing element (e.g. redox moiety) and/or substrate. In one example, the one or more zwitterionic repeating groups comprise a betaine compound or derivative thereof. In one example, the zwitterionic repeating groups are present at the end of the functionalized polymer chain or are present along the backbone of the functionalized polymer chain.

In one example, the functionalized polymer comprises alkanethiol and zwitterionic repeating groups. In one example, the functionalized polymer comprises alkanethiol and betaine groups. In one example, the functionalized polymer comprises mercaptoalkanol and zwitterionic repeating groups. In one example, the functionalized polymer comprises mercaptoalkanol and betaine groups. In one example, the functionalized polymer comprises arylthiol and zwitterionic repeating groups. In one example, the functionalized polymer comprises arylthiol and betaine groups. In one example, the functionalized polymer comprises arylmercaptoalkanol and zwitterionic repeating groups. In one example, the functionalized polymer comprises arylmercaptoalkanol and betaine groups. In one example, the functionalized polymer comprises benzylthiol and zwitterionic repeating groups. In one example, the functionalized polymer comprises benzylthiol and betaine groups. In one example, the functionalized polymer comprises benzylmercaptoalkanol and zwitterionic repeating groups. In one example, the functionalized polymer comprises benzylmercaptoalkanol and betaine groups.

In one example, the APL is physically or chemically coupled to at least a portion of the substrate surface. In one example, the APL is physically or chemically coupled to at least a portion of the substrate surface, with the one or more aptamer conjugates physically or chemically coupled to at least a portion of the substrate surface. In one example, the APL is physically or chemically coupled to at least a portion of the substrate surface, with the one or more aptamer conjugates physically or chemically coupled to at least a portion of the substrate surface, and a substantial remainder of the substrate surface further comprising a physically or chemically coupled co-adsorbate.

In one example, the APL provides sufficient free volume so as to allow reversible conformational change of the one or more aptamer conjugates. In one example, at least one of the one or more aptamer conjugates is physically or chemically coupled to an amino alkanoic acid.

In one example, the one or more aptamer conjugates are present, for example, at densities of 10⁻⁹, 10⁻¹⁰, 10⁻¹¹, 10⁻¹², to 10⁻¹³ molecules/cm² of substrate surface. Other densities can be used. In one example, the aptamers present at the substrate surface have substantially similar architecture (less than 2 base pair deviation), identical transducing elements or redox moieties, identical conjugate coupling chemistries attaching the aptamer to the working electrode surface, SAM, or co-adsorbate, identical aptamer/co-adsorbate mass ratios and/or densities, and identical manufacturing history. In one example, the aptamers present at the substrate surface have different architecture (more than 2 base pair deviation), different or the same transducing elements or redox moieties, different or identical conjugate coupling chemistries attaching the aptamer to the working electrode surface, SAM, or co-adsorbate, different aptamer/co-adsorbate mass ratios and/or densities, and different or identical manufacturing history.

### Signal Transducing Element

In one example, the signal transducing element comprises a redox moiety. The redox moiety may comprise any compound that upon a change in its proximity to an electrode at a biased potential causes a change in electron transfer kinetics. Exemplary redox species include methylene blue, organometallic redox moieties, ferrocene, viologen, anthraquinone or any other quinones, ethidium bromide, daunomycin, metallic porphyrin complexes, crown ether metallic complexes, bis-pyridine metal complexes, bis-imidizole metal complexes, tris-pyridine metal complexes, ethylenetetracetic acid (EDTA)-metal complexes, and cytochromes. In one example, the reversible redox moiety comprises iron, iridium, ruthenium, osmium, a thiazine dye, or derivative thereof. In one example, the reversible redox moiety comprises ferrocene or methylene blue.

In one example, the sensor is configured for continuous, semi-continuous, sequential, or random signal acquisition. In one example, the sensor is configured for transcutaneous insertion.

### Co-Adsorbents

In one example, the presently disclosed AB or EAB device includes one or more co-adsorbents. Co-adsorbents function to cover the substrate (adsorbent) and alter the response to of the substrate to exposure of the surrounding environment such that undesired activity or reactions are eliminated or reduced. The effectiveness of the co-adsorbate can be experimentally measured, for example, by tracking baseline current levels when the substrate is biased with electrical potential. In one example, co-adsorbents are configured to, independently, provide a surface energy modulation environment, a phase separation modulation environment, and/or an intermolecular interaction modulation environment between co-adsorbate molecules and/or co-adsorbate and aptamer molecules, aptamer 102 and substrate 110 surface, any monolayers 103, and/or aptamer protective material 105.

In one example, the one or more co-adsorbents provide, independently, an ionic strength, and an amount of the one or more co-adsorbents is present capable of modulating or maintaining the ionic strength in proximity to the at least one aptamer conjugates and/or the substrate surface.

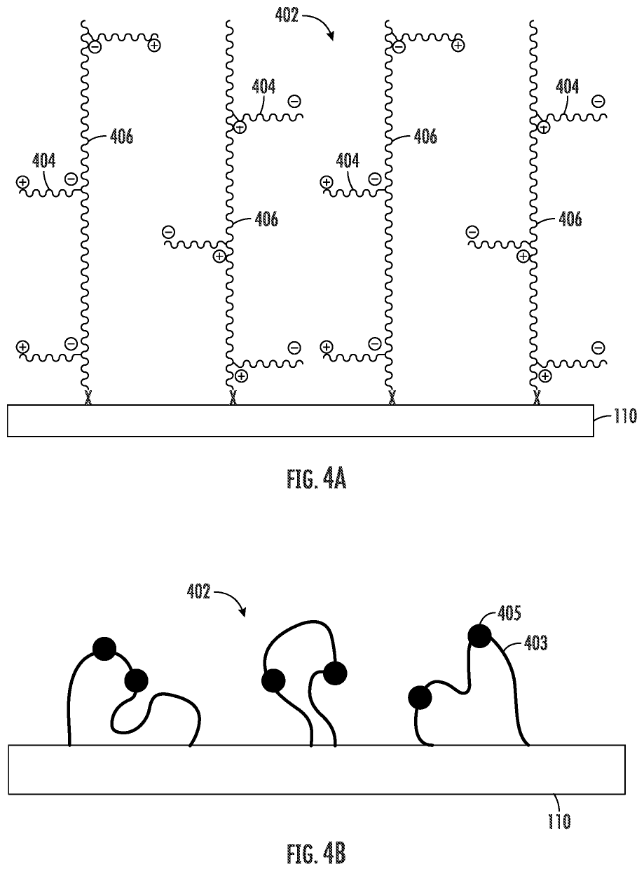

In one example, at least a portion of substrate 110 surface further comprises one or more co-adsorbents. In one example, the one or more co-adsorbents independently comprises a plurality of functional groups. FIGS. 4A and 4B depict schematic representations of exemplary co-adsorbents 402a, and 402b, respectively, in accordance with the broadest aspect of the present disclosure. Thus, FIG. 4A, shows an enlarged cross-sectional schematic of substrate 110 WE surface (as working electrode) of an implantable-type EAB with an exemplary architecture of co-adsorbate 402a having a linear segment 406a and end-group segments 404a. FIG. 4B shows an enlarged cross-sectional schematic of substrate 110 WE surface (as working electrode) of an implantable-type EAB with an exemplary architecture of co-adsorbate 402a having a linear segment 406b and backbone segments 404b. Linear segment 406a, 406b can comprise alkyl, alkylthiol, phenylthiol, benzylthiol, arylthiol, mercaptoakanol, alkylsilane, aromatic silane, or alkylaromatic silane, for example, as disclosed herein. In one example, linear segment 406a, 406b comprises an aromatic thiol, alkylaromatic thiol. In one example, end-group segments 404a or backbone segments 404b can be zwitterionic or repeating zwitterionic groups as disclosed herein. Substrate 110 can comprise a random and/or patterned combination of co-adsorbents 402a and 402b at various substrate surface area ratios. Co-adsorbate 402a, 402b can be coupled to substrate 110 surface (indicated by “X” in FIG. 4A, 4B) in a variety of ways, for example, a thiol, amine, amino, carboxyl, carboxylamine, or carboxylamino, via EDC/NHS chemistry, for example, click chemistry, as discussed herein. FIG. 4C shows an enlarged cross-sectional schematic of substrate 110 WE surface (as working electrode) of an implantable-type EAB with an exemplary architecture of co-adsorbate 403 having linear segment 406 and a linear section 405, where linear segment 406 can be alkyl, alkylthiol, mercaptoakanol for example, as disclosed herein, and linear section 405 can be zwitterionic or repeating zwitterionic groups as disclosed herein. Co-adsorbate 403 can be coupled to substrate 110 surface in a variety of ways, for example, a thiol, amine, amino, carboxyl, carboxylamine, or carboxylamino, via EDC/NHS chemistry, or click chemistry, for example, as discussed herein. Substrate 110 surface can comprise a random and/or patterned combination of co-adsorbents 402a, 402b, and 403 at various substrate surface area ratios.

In one example, the continuous monitoring AB or EAB of the present disclosure comprises one or more co-adsorbents associated with substrate 110 surface, the one or more co-adsorbents being chemically different.

In one example, the aforementioned functionalized APL's can in part, also function as a co-adsorbate. Thus, in one example, at least a portion of the substrate surface comprises the functionalized APL, at least a portion of the substrate surface comprises the one or more co-adsorbents, and at least a portion of a remainder of the substrate surface comprises the one or more aptamer conjugates, where the sum of the percent portions of the substrate surface and the remainder can be 100 percent or less than 100 percent.

In one example, at least a portion of the substrate surface the one or more co-adsorbents and a portion of a remainder of the substrate surface comprises the one or more aptamer conjugates. In one example, the remainder of the substrate surface is about 50% of the total surface area of the substrate. In one example, the remainder of the substrate surface is less than 50% but more than 0% of the total surface area of the substrate. In one example, the remainder of the substrate surface is greater than 50% and less than 100% of the total surface area of the substrate.

In one example, at least a portion of the substrate surface comprises the one or more co-adsorbents and a portion of a remainder of the substrate surface comprises the one or more aptamer conjugates physically or chemically coupled to the substrate. In one example, the one or more co-adsorbents are physically or chemically coupled to the substrate surface and a portion of a remainder of the substrate surface comprises the one or more aptamer conjugates physically or chemically coupled to at least a portion of the co-adsorbate.

In one example, the co-adsorbate comprises a self-assembled monolayer (SAM). In one example, the co-adsorbate comprises a mono-functional or a multi-functional alkanethiol.

In one example, a thiol functional group of the mono-functional alkanethiol or the multi-functional alkanethiol is covalently coupled to at least a portion of the substrate surface. In one example, a thiol functional group of the mono-functional alkanethiol or the multi-functional alkanethiol is covalently coupled to a gold substrate surface.

In one example, the co-adsorbate comprises a mono-functional or a multi-functional mercaptoalkanol. In one example, a thiol functional group of the mono-functional or the multi-functional mercaptoalkanol is covalently coupled to at least a portion of the substrate surface. In one example, a thiol functional group of the mono-functional or the multi-functional mercaptoalkanol is covalently coupled to at least a portion of a gold substrate surface.

In one example, the co-adsorbate comprises a zwitterionic repeating group associated with at least a portion of the substrate surface. In one example, the co-adsorbate comprises a zwitterionic repeating group coupled to at least a portion of the substrate surface. In one example, the co-adsorbate comprises a zwitterionic repeating group covalently to at least a portion of the substrate surface. In one example, the zwitterionic repeating group comprises a betaine group, e.g., a sulfobetaine or carboxybetaine group.

In one example, the zwitterionic repeating group comprises ammoniophosphates or lecithin analogs, ammoniophosphonates, ammoniophosphinates, ammoniosulfonates, ammoniosulfates, ammoniocarboxylates, or combinations thereof.

In one example, the zwitterionic repeating group comprises an alkanethiol betaine. In one example, the alkanethiol is linear and comprises a plurality of betaine groups along its chain. In one example, the alkanethiol is an end-terminated mono-or dithiol with at least one betaine group along its chain. In one example, the alkanethiol is linear and comprises an end-terminated betaine group. In one example, a thiol group of the end-terminated dithiol alkanethiol is covalently coupled to the substrate surface.

In one example, the zwitterionic repeating group comprises an mercaptoalkanol betaine. In one example, the mercaptoalkanol is linear and comprises a plurality of betaine groups along its chain. In one example, the mercaptoalkanol is linear and comprises an end-terminated betaine group. In one example, a thiol group of the mercaptoalkanol is covalently coupled to the substrate surface.

In one example, the co-adsorbate is one or more of the following structures:

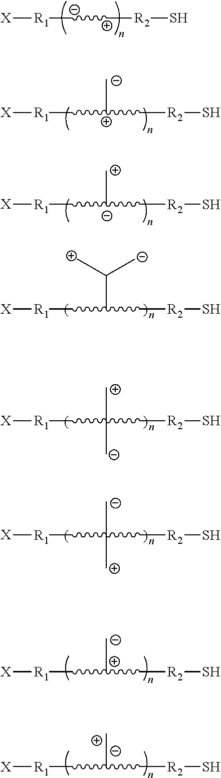

where “ [symbol] ” represents a hydrocarbon chain; where a zwitterionic unit is connected to a backbone and the charges are on side-groups that are pendant to the backbone, or the zwitterionic unit is such that one or both charges is on the backbone; where R1 and R2 are, independently, branched or unbranched acyclic alkyl, substituted or unsubstituted cyclic alkyl, substituted or unsubstituted aryl, substituted or unsubstituted benzyl, substituted or unsubstituted heteroalkyl, substituted or unsubstituted heterocyclic; where X is —OH, —NHR1, —NH2, or —SH; where n is an integer of 2 to about 1000; or

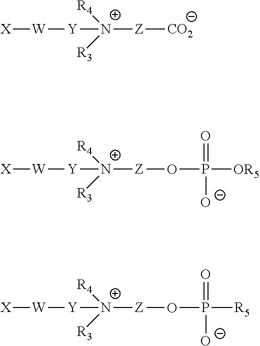

where X is —OH, —NHR1, —NH2, or —SH; where W, Y, and Z are, independently, branched or straight chain alkyl, heteroalkyl, cycloalkyl, cycloheteroalkyl, aryl, or heteroaryl, any of which can be optionally substituted with O, OH, halogen, amido, or alkoxyl; R1 is H, branched or unbranched acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl; and R3, R4, and R5, are independently chosen from acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl.

In one example, the co-adsorbate is one or more of the following structures:

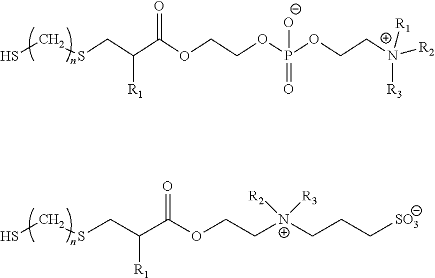

where R1 is H, alkyl, heteroalkyl, cycloalkyl, cycloheteroalkyl, aryl, or heteroaryl; and R2, R3, and R4, are independently chosen from alkyl, heteroalkyl, cycloalkyl, cycloheteroalkyl, aryl, or heteroaryl; n is an integer from 2-24.

In one example, the co-adsorbate is one or more of the following ammoniosulfonates (sulfobetaines) or ammoniosulfates, structures:

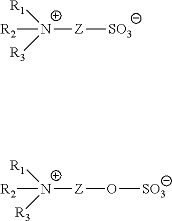

where Z is branched or straight chain alkyl, heteroalkyl, cycloalkyl, cycloheteroalkyl, aryl, or heteroaryl; R1 is H, alkyl, heteroalkyl, cycloalkyl, heterocycloalkyl, aryl, or heteroaryl; and R2 and R3, are independently chosen from alkyl, heteroalkyl, cycloalkyl, heterocycloalkyl, aryl, or heteroaryl; wherein one or more of R1, R2, R3, and Z are substituted with a polymerization group; and ammoniocarboxylates having the structures:

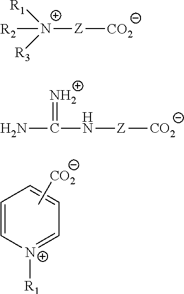

where Z is branched or straight chain alkyl, heteroalkyl, cycloalkyl, cycloheteroalkyl, aryl, or heteroaryl; R1 is H, alkyl, heteroalkyl, cycloalkyl, heterocycloalkyl, aryl, or heteroaryl; and R2 and R3 are independently chosen from alkyl, heteroalkyl, cycloalkyl, heterocycloalkyl, aryl, or heteroaryl; wherein one or more of R1, R2, R3, and Z are substituted with a polymerization group.

In each of these monomers, Z can have a length of from 1 to 12 atoms, e.g., from 1, 2, 3, 4, 5, 6, 7, 8, 9, 10, 11, or 12 atoms, where any of these values can form an upper or lower endpoint of a range.

These compounds or monomers can be prepared by methods known to those of skilled in the art, e.g., as detailed in Laschewsky, “Structures and synthesis of zwitterionic polymers,” Polymers 6:1544-1601, 2014. In certain examples, the disclosed polyzwitterions can have repeating zwitterionic units obtained from any of the zwitterionic compounds or monomers disclosed above. Exemplary zwitterionic compounds include ocamidopropyl betaine, oleamidopropyl betaine, octyl sulfobetaine, caprylyl sulfobetaine, lauryl sulfobetaine, myristyl sulfobetaine, palmityl sulfobetaine, stearyl sulfobetaine, betaine (trimethylglycine), octyl betaine, phosphatidylcholine, glycine betaine, poly (carboxybetaine), poly (sulfobetaine), and derivatives thereof. Exemplary monomers comprising one or more pendent or terminal groups with zwitterionic groups comprising or derived from ocamidopropyl betaine, oleamidopropyl betaine, octyl sulfobetaine, caprylyl sulfobetaine, lauryl sulfobetaine, myristyl sulfobetaine, palmityl sulfobetaine, stearyl sulfobetaine, betaine (trimethylglycine), octyl betaine, phosphatidylcholine, glycine betaine, poly (carboxybetaine), and poly (sulfobetaine).

In one example, the co-adsorbate is an end-terminated mono-or dithiol with at least one zwitterionic groups or zwitterionic repeating groups as disclosed herein. In one example, controlling ionic strength, pH, etc., comprises configuring the APL backbone or one or more appendages from its backbone with one or more zwitterionic betaine groups.

In one example, the APL comprises alkanethiol or (aryl) mercaptoalkanol and one or more zwitterionic groups. In one example, controlling ionic strength, pH, etc., comprises providing the APL in combination with mercaptoalkanol having zwitterionic betaine groups.

### Methods

The presently disclosed APL-EAB constructs provide advantages over EAB's without an APL. For example, the presently disclosed APL-EAB constructs can be used in a method of determining an in vivo concentration of an analyte. For example, the method can comprise the steps of contacting, in vivo, an biological fluid comprising an analyte with the presently disclosed APL-EAB constructs, the EAB coupled to a conductive substrate, the EAB encapsulated in the APL, the APL being permeable to an analyte, and the EAB producing a signal upon interaction with the analyte.

The presently disclosed APL-EAB constructs are configured to receive bias voltage that is varied to reversibly oxidize and reduce a redox probe associated with the aptamer, with the aptamer being associated with a conductive substrate surface. The presently disclosed APL-EAB constructs can be used in a method that comprises interrogating the conductive substrate or the APL-aptamer-redox moiety conjugate. The method can further comprise detecting the signal generated by the aptamer-redox moiety conjugate in the presence of a concentration of analyte and correlating an in vivo concentration of the analyte based on the detected signal, change of signal, difference of signal, etc. In one example, signal transduction by the presently disclosed APL-EAB constructs is determined by electron transfer rate from the reversible redox probe, where the electron transfer rate difference correlates to analyte concentrations.

In one example, the interrogating is continuous, semi-continuous, sequential, or a random temporal detecting of the signal. In one example, the method can further comprise the step of adjusting the signal based on a background signal produced as a result of non-specific binding of the aptamer biosensor so as to produce an adjusted signal. Two or more working electrodes, with or without the presently disclosed APL-EAB constructs can be used. The method can further comprise determining the in vivo concentration of the analyte during a period of time based on the adjusted signal.

In one example, interrogating comprises a differential measurement technique. Exemplary differential measurement techniques include, for example, interrogating with a first square wave voltammetry (SWV) frequency to obtain a first signal and a second SWV frequency to obtain a second signal, taking the difference between the two signals, and dividing by the average of the two signals to obtain an adjusted signal. In one example, the interrogating comprises chronoamperometry. In one example, the interrogating comprises cyclic voltametry.

In one example, the presently disclosed APL's can control or modulate intermolecular interactions between the aptamer conjugate and the APL. In another example, the presently disclosed APL's constructs, alone or in combination with the presently disclosed co-adsorbate(s), provides for reducing decoupling of the aptamer from the substrate surface. In another example, the presently disclosed APL's can control or modulate diffusion of the aptamer from proximity about the substrate surface. Thus, if reversible desorption/decoupling of the aptamer from the substrate occurs, the presently disclosed APLs can keep the aptamer in proximity to the substrate surface so as to increase re-absorption/re-coupling of the aptamer. In one example, the presently disclosed APLs are partially cross-linked. The presently disclosed APL's can be crosslinked in the presence of aptamer-transducing moiety with little or no adverse effect on the APL-EAB performance as discussed below.

In one example, the presently disclosed APLs can be used to extend the in vivo end-of-life for an EAB device. For example, the presently disclosed APLs have demonstrated extended end-of-life in bovine serum albumin for up to 20 hrs. It is envisaged that the presently disclosed APLs can provide EABs with up to one day, 2 days, one week, 2 weeks, 3 weeks, or one month in vivo end-of-life performance.

In one example, the presently disclosed APLs alone in in combination with a SAM or a co-adsorbate, provides for a method to control or modulate the ionic strength about the aptamer-signal transducing element conjugate. For example, presently disclosed functionalized APLs, e.g., betaine functionalized APL's in combination with one or more co-adsorbents can be present in an amount capable of modulating or maintaining the ionic strength about the aptamer-signal transducing element conjugate.

Methods of manufacturing the presently disclosed APL-EAB devices include presenting an aptamer comprising a reversible redox moiety to a surface of a conductive substrate and presenting the APL to the portion of the surface of the conductive substrate so as to encapsulate the aptamer conjugate in the APL. Alternatively, the presently disclosed APL-EABs are combined with the aptamer comprising a reversible redox moiety and presented to the substrate surface.

### Experimental Results

A series of exemplary APL's were developed and tested with an aptamer-redox moiety conjugate EAB constructs and the effectiveness of the APL's in providing improvement in one or more attributes of the constructs evaluated. Characteristics of a representative sampling of the APL's are summarized in Table 1.

TABLE 1
Exemplary APLs.

| | | | | Number Average | |
|---|---|---|---|---|---|
| | Hydrophilic | Functional | Hydrophobic | Molecular | |
| | Segment | Content | Segment | Weight (Mn) | Polydispersity |
| APL | Wt % | Wt % | Wt % | (kDaltons) | Index |
| PUU-3 | 21 | 18 | 27 | 140 | 1.7 |
| PUU-8 | 23 | 27 | 14 | 126 | 1.7 |
| PUU-9 | 27 | 34 | 9 | 132 | 1.7 |
| crosslinked | | | | | |
| PUU-10 | 35 | 40 | 0 | 151 | 1.9 |
| crosslinked | | | | | |
| PU-3 | 28 | 18 | 23 | 102 | 2.4 |
| PU-8 | 29 | 23 | 20 | 111 | 2.1 |

PUU = aliphatic polyurethane urea segmented block copolymer. PU = aliphatic polyurethane segmented block copolymer. Hydrophilic segment = polyethylene glycol and polycarbonate. Hydrophobic segment = poly dimethyl siloxane. Functional content = sulfobetaine or carboxybetaine. Crosslinker = polyglycol polyglycidyl (PEG-PG). Wt % values may vary by +/− 10%.

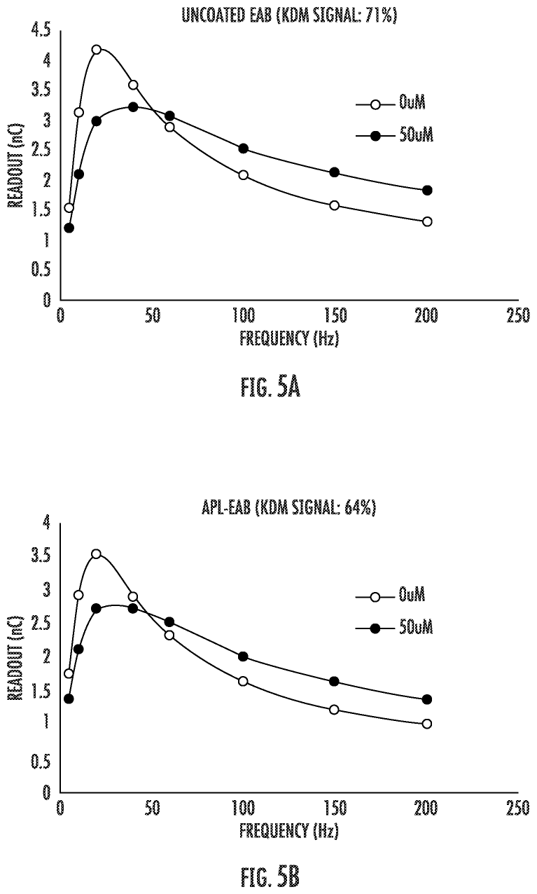

FIGS. 5A and 5B are representative graphs of experimental voltametric readout vs. electron transfer readout (charge vs. frequency) data of an exemplary aptamer biosensor with and without APL. In this example, the co-adsorbate was 6-mercapto-1-hexanol, the coupling chemistry was thiol association on a gold substrate, the APL used was PUU-3.

Aptamers tested were specific for vancomycin and aminoglycosides. FIGS. 5A and 5B demonstrates a kinetic differential measurement (KDM) signal of about 71% with 50 umol/L analyte spike in a pre-serum PBS buffer solution without an APL (“control EAB”), whereas APL coated sample (“APL-EAB”) provides 67% KDM signal under identical conditions.

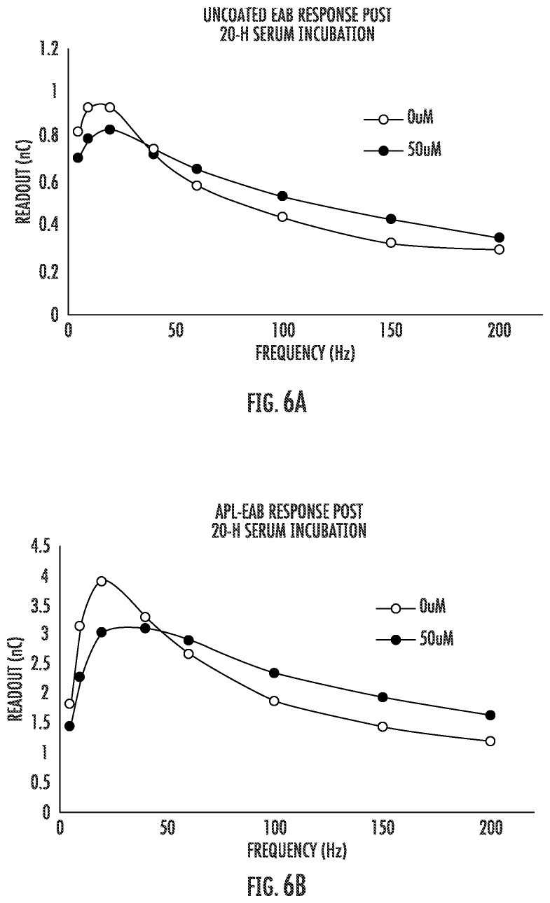

With reference to FIGS. 6A, 6B, samples after 20 hours of exposure to biofluid (50 um serum incubation), demonstrates the APL sample PUU-9 maintains a good KDM while the control shows significant degradation of KDM. The APL sample demonstrates signal of about 4× that of the control after 20 hours.

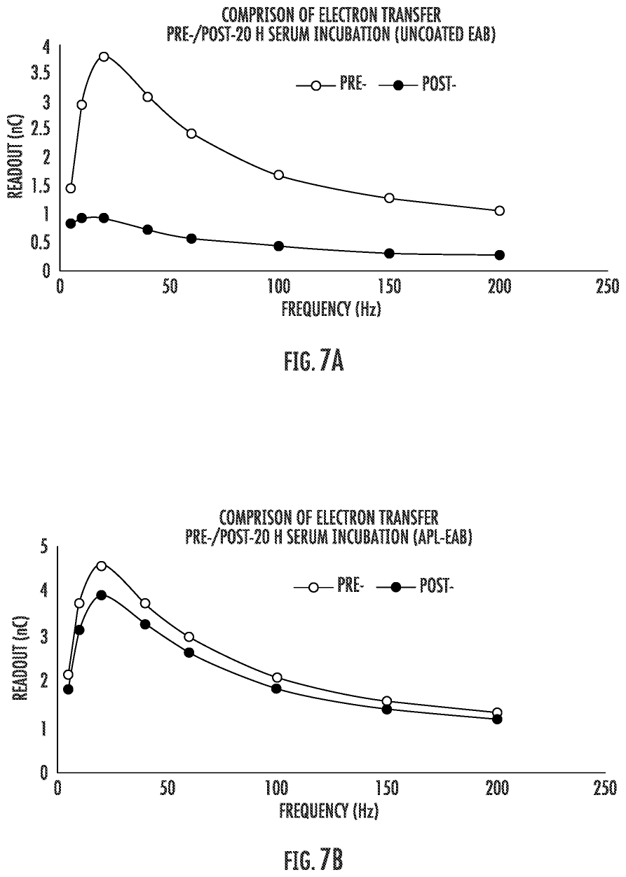

FIGS. 7A and 7B are representative graphs of experimental charge vs. frequency data of protein fouling of the control verse the same aptamer conjugate with aptamer protective material. The data demonstrates the advantage of the APL in maintaining EAB response by resisting biofouling as compared to the uncoated control.

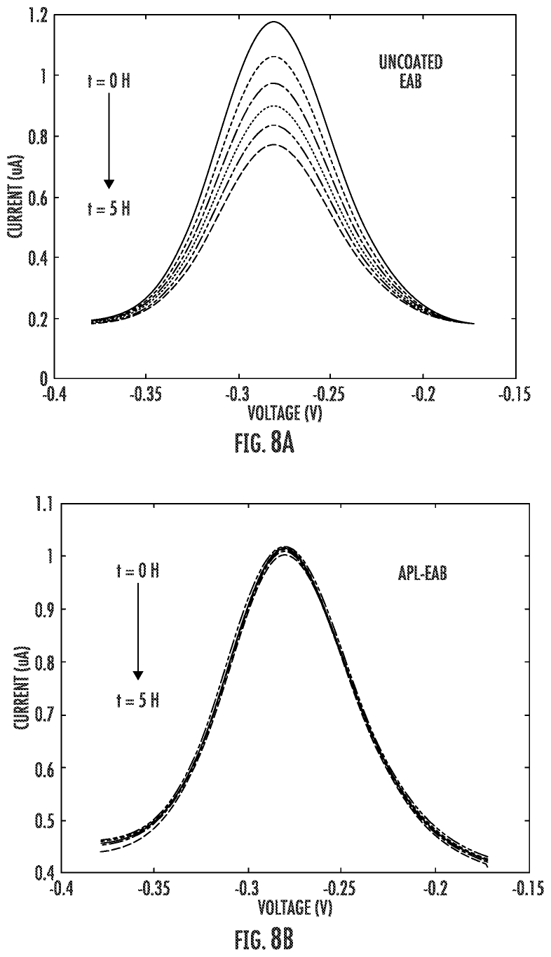

FIGS. 8A and 8B are representative graphs of experimental current vs. frequency voltammogram data taken a different time intervals for an exemplary aminoglycoside aptamer biosensor in a protein-spiked buffer solution without aptamer protective material compared to an APL-aminoglycoside aptamer sample, respectively. The data in FIG. 8A shows the uncoated EAB has a continuous decay in signal output in a protein-containing environment, e.g., a gradual shrinkage of peak height over time, whereas in contrast, the APL aminoglycoside aptamer sample of FIG. 8B show minimal change in readout.

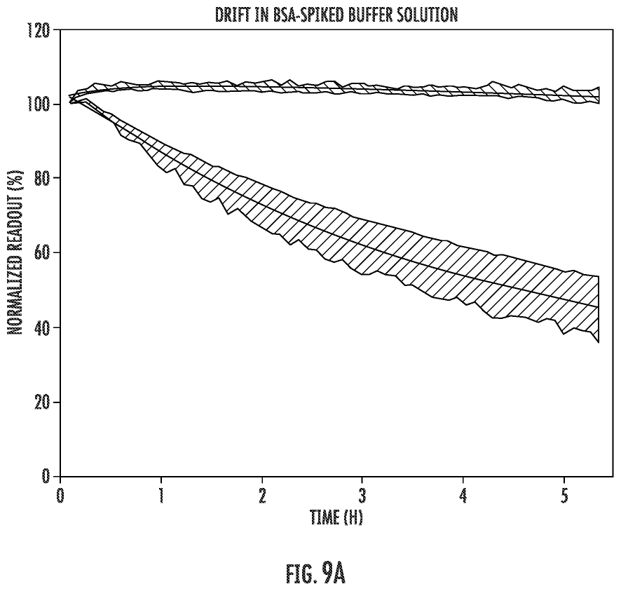

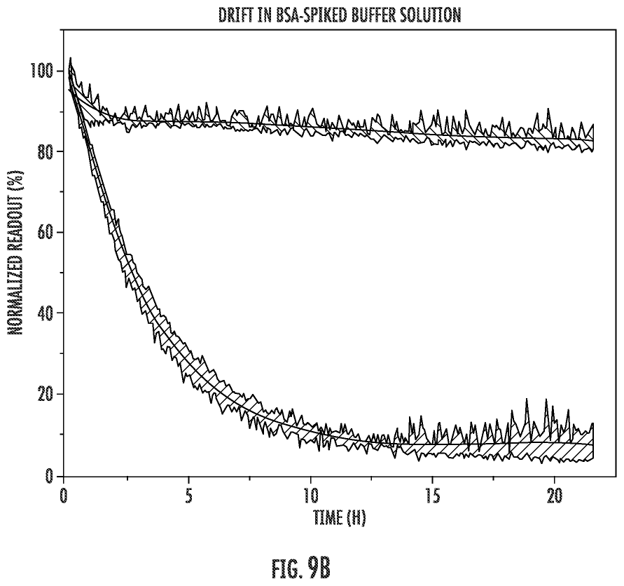

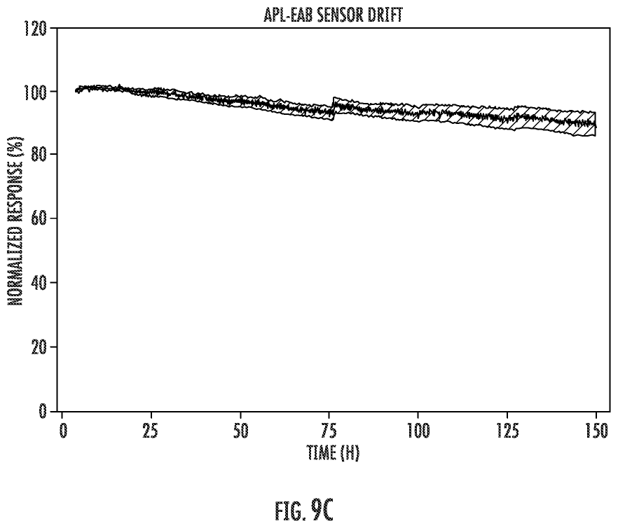

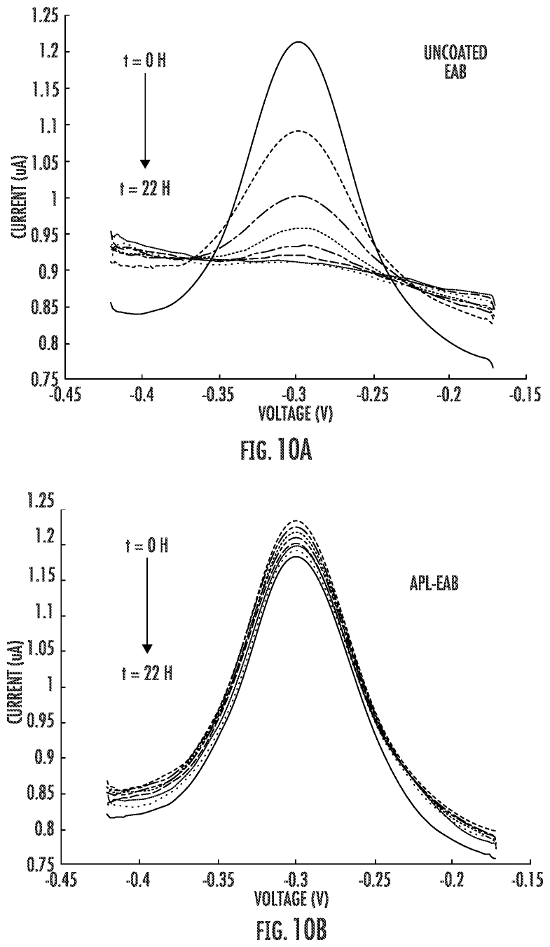

FIG. 9A is a representative graph of experimental normalized readout percentage vs. time representing drift for control EAB's compared with APL EAB's without biofouling challenge. The samples were exposed to bovine serum albumin (BSA) and as shown, the control EABs without the APL began drifting from biofouling etc., after less than 1 hour whereas the APL-EAB's provided stable performance for at least 5 hrs.

FIG. 9B is a representative graph of experimental normalized readout percentage vs. time representing drift for control EAB's compared with APL-EAB's in a buffer solution comprising biofouling protein. The samples were exposed to bovine serum albumin (BSA), and as shown, the control EABs without the APL began drifting from biofouling etc., after less than 1 hour whereas the APL-EAB's, for example, sample PUU-9, provided stable performance for at least 22 hours (no analyte challenge). Thus, a functional content of zwitterion present in at least 10 wt. % in a PU or PUU provides for one or more performance improvements for EAB's, e.g., calibration stability, storage stability, drift stability, localized pH stability, and interference reduction.

FIG. 9C is a representative graph of experimental normalized readout percentage vs. time representing stability for an exemplary APL EAB sensor PUU-3 in PBS at 37° C. As shown, the APL-EAB sample demonstrated stability for at least 6 days with at least 80% of signal remaining. This data demonstrates the stability enhancement properties of the presently disclosed APLs.

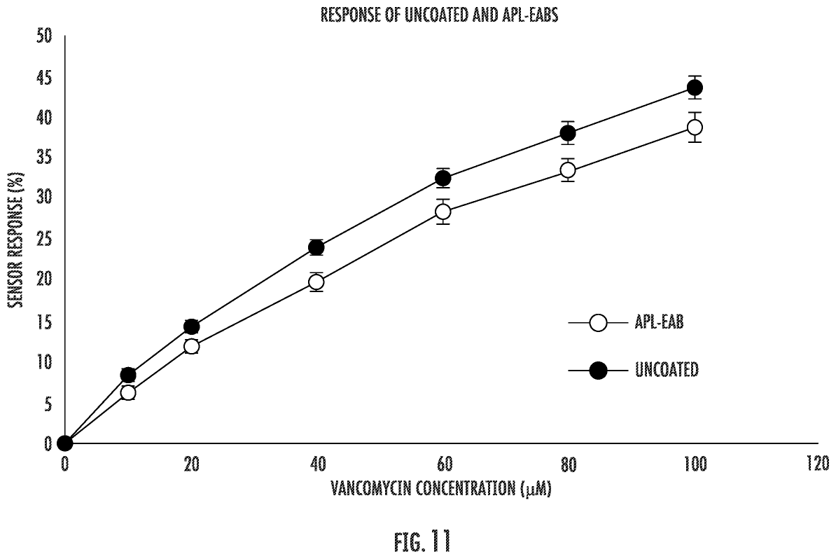

FIGS. 10A and 10B are representative graphs of experimental current vs. frequency voltammogram data for an exemplary vancomycin aptamer biosensor without aptamer protective material compared to a an APL-vancomycin aptamer sample, respectively. Again, the data shows the uncoated EAB has a continuous decay in signal output during potential cycling, whereas the APL vancomycin aptamer sample is significantly more robust.

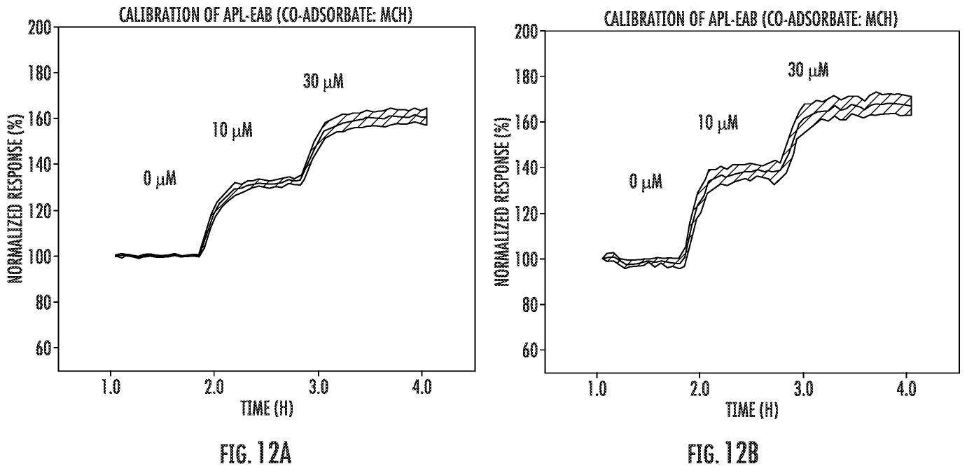

With reference to FIG. 11, a representative graph of experimental sensor response percentage vs. analyte concentration of an exemplary EAB with and without aptamer protective material exposed to various analyte concentrations is shown. Vancomycin EAB with APL (PUU-3) provided a signal-concentration curve essentially equivalent to a vancomycin EAB without APL, though the sensor response was slightly lower at a given Vancomycin concentration.

In the aforementioned experiments, substrates used were a standard disc gold electrode. Follow up studies with other electrode form factors indicated that the present EAB-APL's are not substrate form-dependent and can been replicated, for example, using a wire electrode form factor. Electrode form factor does affect the absolute signal level, do to at least, differences in surface area.

FIGS. 12A and 12B are representative calibration graphs of for an exemplary vancomycin aptamer biosensor with different co-adsorbents, 6-mercapto-1-hexanol (MCH) and 8-mercapto-1-hexanol (MCO), respectively, challenged with 0 uM, 10 uM, and 30 uM concentration of analyte. The data from FIGS. 12A and 12B demonstrate good calibration and compatibility of various co-adsorbents with the presently disclosed APLs.

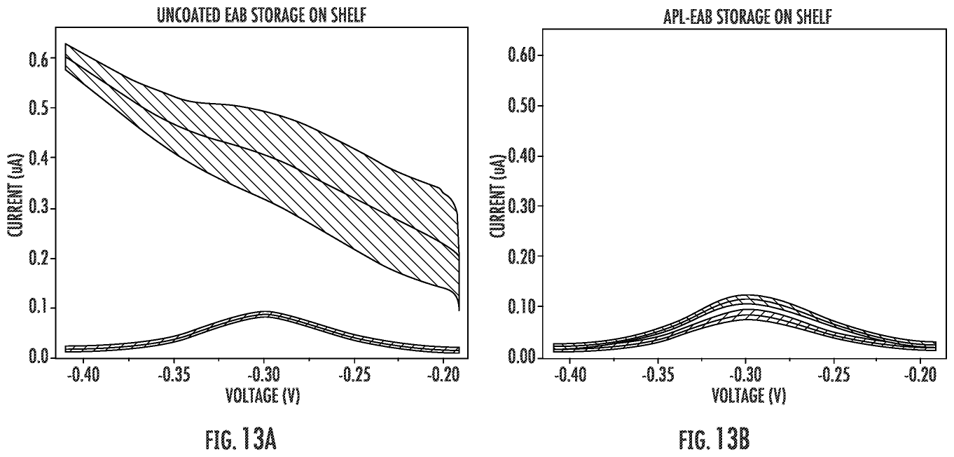

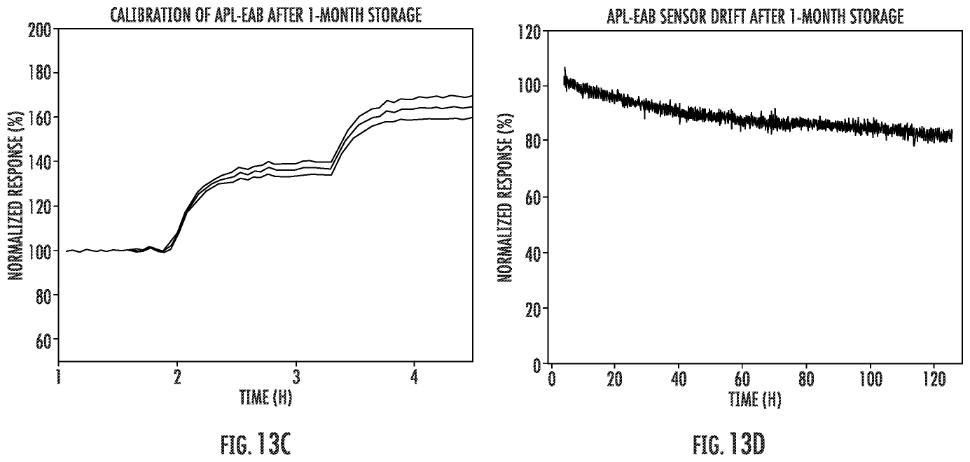

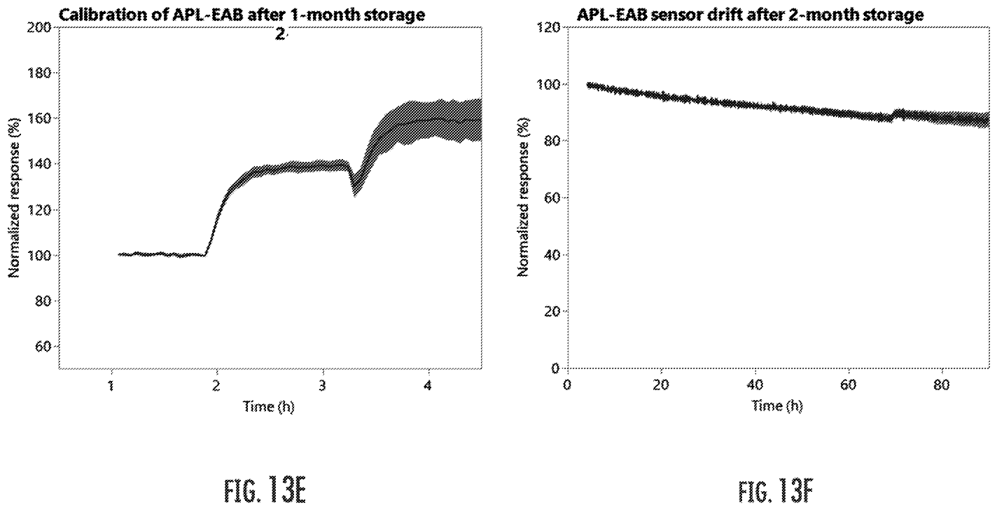

FIGS. 13A and 13B are representative graphs of shelf-life performance uncoated vs APL-coated EABs, respectively, after storage for 5 h in ambient environment. Uncoated sensor showed significant performance degradation after storage, with large background current, while minimal change in performance was observed on APL-EAB.

FIGS. 13C and 13D are representative graphs of calibration and drift data of an exemplary APL-EAB (targeting vancomycin) after 1 month storage in an ambient air environment at room temperature and relative humidity, in the dark. FIGS. 13E and 13F are representative graphs of calibration and drift data of an exemplary APL-EAB (targeting vancomycin) after 2 months storage in an ambient air environment at room temperature and relative humidity, in the dark. The data from FIGS. 13C-13F demonstrate good calibration and drift performance over at least two months using the presently disclosed APLs that employ an APL and co-adsorbent as presently disclosed.

### Drug Releasing Layer

Devices and probes that are transcutaneously inserted or implanted into subcutaneous tissue conventionally elicit a foreign body response (FBR), which includes invasion of inflammatory cells that ultimately forms a foreign body capsule (FBC), as part of the body's response to the introduction of a foreign material. The continuous monitoring systems discussed herein include continuous analyte monitoring systems configured to monitor one, two, or more analytes concurrently, sequentially, and/or randomly (which is inclusive of events that can take place independently in picoseconds, nanoseconds, milliseconds, seconds, or minutes) to predict health-related events and health systems performance (e.g., the current and future performance of the human body's systems such as the circulatory, respiratory, digestive, or other systems or combinations of organs or systems). In examples, insertion or implantation of a device, for example, an EAB sensing device, can result in an acute inflammatory reaction resolving to chronic inflammation with concurrent building of fibrotic tissue, such as described in detail above. Eventually, over a period of time, a mature FBC, including primarily contractile fibrous tissue forms around the device. See Shanker and Greisler, Inflammation and Biomaterials in Greco RS, ed., “Implantation Biology: The Host Response and Biomedical Devices” pp 68-80, CRC Press (1994). The FBC surrounding conventional implanted devices has been shown to hinder or block the transport of analytes across the device-tissue interface. Thus, continuous extended life analyte transport (e.g., beyond the first few days) in vivo has been conventionally believed to be unreliable or impossible.

In some examples, certain aspects of the FBR in the first few days may play a role in noise. It has been observed that some sensors function more poorly during the first few hours after insertion than they do later. This is exemplified by noise and/or a suppression of the signal during the first few hours (e.g., about 2 to about 24 hours) after insertion. These anomalies often resolve spontaneously after which the sensors become less noisy, have improved sensitivity, and are more accurate than during the early period. It has been observed that some transcutaneous sensors and wholly implantable sensors are subject to noise for a period of time after application to the host (i.e., inserted transcutaneously or wholly implanted below the skin).

Thus, with reference back to FIG. 2D, a drug releasing layer, membrane, matrix, or coating 113 can be positioned adjacent or directly adjacent the APL 105 in one example the presently disclosed AB or EAB continuous sensor comprises an immune response attenuating layer or drug releasing layer configured to interact with the immune system of a host or release an active agent into the environment of the sensor. In one example, the immune response attenuating layer includes an active agent that is coupled to or entrapped in the layer, e.g., covalently coupled active agent (a dexamethasone derivative or analog) or a surface exposed, active agent (e.g., silver nanoparticles). In one example, the drug releasing layer comprises an active agent that is configured to release from the layer over time to mitigate or attenuate an immune response. Such drug releasing layers include, for example, segmented polyurethane polymers containing dexamethasone and/or dexamethasone acetate and/or other dexamethasone derivative or analog as disclosed in co-assigned U.S. application Ser. No. 17/945,585, which is incorporated herein by reference.

### Manufacturing

The substrate can by formed by a variety of manufacturing techniques (bulk metal processing, deposition of metal onto the substrate, or the like). In one example, the substrate is plated wire (e.g., platinum on steel wire) or bulk metal (e.g., gold wire). It is believed that substrates for EAB's formed from bulk metal wire provide superior performance (e.g., in contrast to deposited electrodes), including increased stability of assay, simplified manufacturability, resistance to contamination (e.g., which can be introduced in deposition processes), and improved surface reaction (e.g., due to purity of material) without peeling or delamination. The substrate can be a metal wire with an outer insulator. The substrate can be a plurality of metal wires each with an outer insulator.

In examples wherein an outer insulator is disposed about a substrate, a portion of the coated assembly structure can be stripped or otherwise removed, for example, by hand, excimer lasing, chemical etching, laser ablation, grit-blasting (e.g., with sodium bicarbonate, solid carbon dioxide, or other suitable grit), or the like, to expose the electrochemically active surfaces. Alternatively, a portion of the electrode can be masked prior to depositing the insulator in order to maintain an exposed electrochemically active surface area. In one exemplary example, grit blasting is implemented to expose the electrochemically active surfaces, preferably utilizing a grit material that is sufficiently hard to ablate the polymer material, while being sufficiently soft so as to minimize or avoid damage to the underlying metal electrode (e.g., a platinum electrode). Although a variety of “grit” materials can be used (e.g., sand, talc, walnut shell, ground plastic, sea salt, solid carbon dioxide, and the like), in some one example, sodium bicarbonate is an advantageous grit-material because it is sufficiently hard to ablate, e.g., a parylene coating without damaging, e.g., an underlying platinum conductor. One additional advantage of sodium bicarbonate blasting includes its polishing action on the metal as it strips the polymer layer, thereby eliminating a cleaning step that might otherwise be necessary. Etching (chemical or plasma, for example) or other methods can be used to provide nanopores and/or micropores to the substrate surface.

In some examples, a radial window is formed through the insulating material to expose a circumferential electrochemically active surface of the working electrode. Additionally, sections of electrochemically active surface of the reference electrode are exposed. For example, the sections of electrochemically active surface can be masked during deposition of an outer insulating layer or etched after deposition of an outer insulating layer.

In examples, the APL is deposited on the substrate comprising the aptamer conjugate to yield a domain thickness of from about 0.05 micron or less to about 40 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more microns., In one example, a domain thickness of APL is from about 20 microns to about 40 microns, including all ranges and subranges therebetween. In one example, the APL is deposited together with the aptamer conjugate. In one example, the APL (or APL and aptamer conjugate) is deposited by spray coating or dip coating. The spraying process atomizes and mists the solution, and therefore most or all of the solvent is evaporated prior to the coating material settling on the underlying domain, thereby minimizing contact of the solvent with the aptamer. While not wishing to be bound by theory, it is believed that during the process of depositing the APL as described in the present disclosure, a structural morphology is formed about the aptamer that allows for substantially unimpeded conformational change by the aptamer with target analyte do to the hard-soft multi-segmented and/or functionalization of the APL structure.

In examples, the APL is deposited on the substrate/co-adsorbant by spray-coating a solution of from about 1 wt. % to about 5 wt. % polymer and from about 95 wt. % to about 99wt. % solvent, including all ranges and subranges therebetween. In spraying a solution of APL material, including a solvent, onto the substrate/co-adsorbant, it is desirable to mitigate or substantially reduce any contact with aptamer of any solvent in the spray solution that can deactivate the underlying aptamer, transducing element, or redox moiety. One or more solvents, including water, can be used, as is appreciated by one skilled in the art.

Although a variety of spraying or deposition techniques can be used, spraying the APL material and rotating the sensor at least one time by 180° can provide adequate coverage by the APL. Spraying the APL material and rotating the sensor at least two times by 120 degrees provides even greater coverage (one layer of 360° coverage), thereby ensuring protection to the AB or EAB, such as is described in more detail above.

In examples, the APL is spray-or dip-coated and subsequently cured (e.g., if crosslinker is used) for a time of from about 15 to about 90 minutes at a temperature of from about 40 to about 60° C. (and can be accomplished under vacuum (e.g., 20 to 30 mmHg)), including all ranges and subranges therebetween. A cure time of up to about 90 minutes or more can be advantageous to ensure complete drying of the APL. While not wishing to be bound by theory, it is believed that complete drying of the APL aids in stabilizing the sensitivity of the AB or EAB sensor signal. It reduces drifting of the signal sensitivity over time, and complete drying is believed to stabilize performance of the AB or EAB sensor signal.

In examples, the APL is formed by spray-or dip-coating one or more layers (e.g., rotating the sensor by 120° for 360° coverage) and optionally curing at 50° C. under vacuum for 60 minutes. However, the APL can be formed by dip-coating, depending upon the concentration of the solution, insertion rate, dwell time, withdrawal rate, and/or the desired thickness of the resulting APL. In one example, the APL and/or the aptamer conjugate is combined with one or more antioxidants. In one example, antioxidants are incorporated in the aptamer protection layer (APL). In one example, antioxidants are incorporated in the aptamer protection layer (APL) in an amount of about 0.01 wt. % to about 5 wt. %. In one example, antioxidants incorporated in the aptamer protection layer (APL) reduce oxidation thiol moieties of the aptamer conjugate or thiol-gold bonds, thus increase the operation lifetime and/or shelf life of the presently disclosed EAB. In one example, the antioxidant is lipophilic, for example Vitamin E or other tocopherols. In one example, the antioxidant is butylated hydroxytoluene (BHT). In one example, the antioxidant is directly added into the APL polymer solution (provisional patent submitted) and deposited onto the aptamer conjugate, for example, using multi-dip processes.

In another example, the antioxidant is hydrophilic, such as ascorbic acid, trehalose, or sodium bisulfite, that is coated as a separate layer or introduced between the aptamer conjugate and APL. In one example, the antioxidant is grafted onto the APL or grafted toa polymer chain that is miscible or compatible with the APL.

### Electronics

In one example, the presently disclosed continuous AB or EAB sensor further comprises one or more of a transmitter, receiver, controller, or power supply. Any electronics associated with continuous analyte sensors, such as non-invasive, minimally invasive, and/or invasive (e.g., transcutaneous and wholly implantable) sensors is applicable. For example, the sensor electronics and data processing as well as transceiver electronics, Wi-Fi, Bluetooth, RF and data processing known in the art can be incorporated into the presently disclosed AB or EAB sensor.

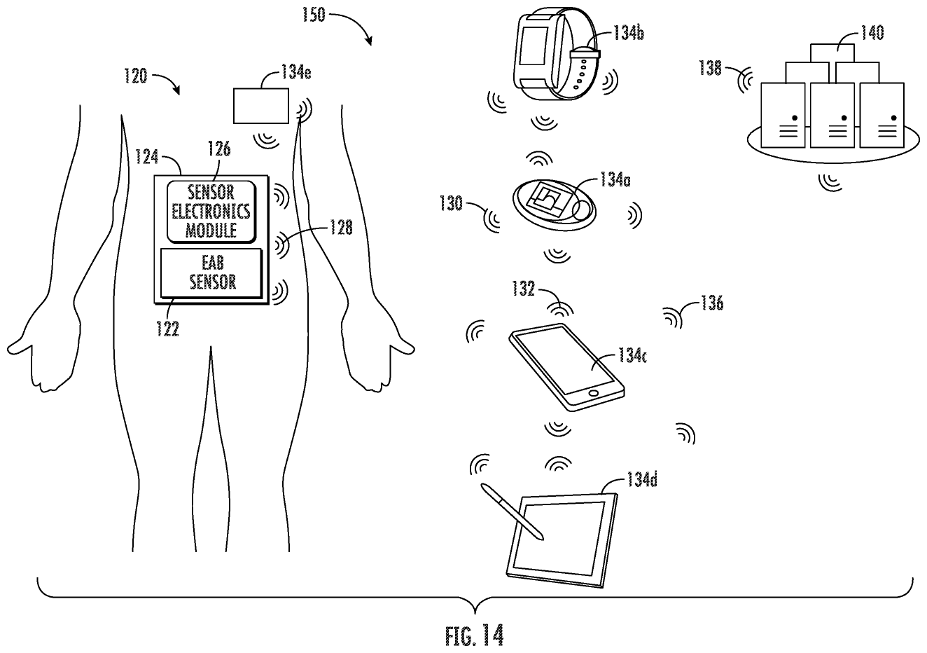

FIG. 14 is a diagram depicting an example continuous AB or EAB system 150 configured to measure one or more analytes alone or in combination with electrophysiological indicators (e.g., blood pressure, heart rate, core temperature, etc.) as discussed herein. The continuous AB or EAB system 150 includes exemplary continuous AB or EAB device 100, 200 operatively connected to a host 120 and a plurality of display devices 134a-e according to certain aspects of the present disclosure. It should be noted that display device 134e alternatively or in addition to being a display device, may be a medicament delivery device that can act cooperatively with the continuous AB or EAB system 150 to deliver medicaments to host 120. In one example, the continuous AB or EAB system 150 is an EAB system, where a sensor electronics module 126 and a continuous EAB sensor 122 associated with the sensor electronics module 126. The sensor electronics module 126 may be in direct wireless communication with one or more of the plurality of the display devices 134a-e via wireless communications signals. In one example, display devices 134a-e may also communicate amongst each other and/or through each other to continuous AB or EAB system 150. For ease of reference, wireless communications signals from analyte sensor system 124 to display devices 134a-e can be referred to as “uplink” signals 128. Wireless communications signals from, e.g., display devices 134a-e to continuous AB or EAB system 150 can be referred to as “downlink” signals 130. Wireless communication signals between two or more of display devices 134a-e may be referred to as “crosslink” signals 132. Additionally, wireless communication signals can include data transmitted by one or more of display devices 134a-d via “long-range” uplink signals 136 (e.g., cellular signals) to one or more remote servers 140 or network entities, such as cloud-based servers or databases, and receive long-range downlink signals 138 transmitted by remote servers 140.

The sensor electronics module 126 includes sensor electronics that are configured to process sensor information and generate transformed sensor information. In certain examples, the sensor electronics module 126 includes electronic circuitry associated with measuring and processing data from continuous EAB sensor 122, including prospective algorithms associated with processing and calibration of the continuous analyte sensor data. The sensor electronics module 126 can be integral with (non-releasably attached to) or releasably attachable to the continuous EAB sensor 122 achieving a physical connection therebetween. The sensor electronics module 126 may include hardware, firmware, and/or software that enables analyte level measurement. For example, the sensor electronics module 126 can include a potentiostat, a power source for providing power to continuous EAB device 122, other components useful for signal processing and data storage, and a telemetry module for transmitting data from itself to one or more display devices 134a-e. Electronics can be affixed to a printed circuit board (PCB), or the like, and can take a variety of forms. For example, the electronics can take the form of an integrated circuit (IC), such as an Application-Specific Integrated Circuit (ASIC), an electrochemical analog front end, a microcontroller, and/or a processor. In one example, the electrochemical analog front end is configured with a sequencer or waveform synthesizer to create the appropriate waveforms to transduce the signal from the EAB. Exemplary waveforms include squarewave voltammetry, linear sweep voltammetry, cyclic voltammetry, differential pulse voltammetry, AC voltammetry, pulse voltammetry, staircase voltammetry, normal pulse voltammetry, chronoamperometry, and chronocoulometry. Examples of systems and methods for processing sensor analyte data are described in more detail in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966and 2007/0208245, each of which are incorporated herein by reference in their entirety for all purposes.

Display devices 134a-e are configured for displaying, alarming, and/or basing medicament delivery on the sensor information that has been transmitted by the sensor electronics module 126 (e.g., in a customized data package that is transmitted to one or more of display devices 134a-e based on their respective preferences). Each of the display devices 134a-e can include a display such as a touchscreen display for displaying sensor information to a user (most often host 120 or a care taker/medical professional) and/or receiving inputs from the user. In some examples, the display devices 134a-e may include other types of user interfaces such as a voice user interface instead of or in addition to a touchscreen display for communicating sensor information to the user of the display device 134a-e and/or receiving user inputs. In some examples, one, some or all of the display devices 134a-e are configured to display or otherwise communicate the sensor information as it is communicated from the sensor electronics module 126 (e.g., in a data package that is transmitted to respective display devices 134a-e), without any additional prospective processing required for calibration and real-time display of the sensor information.

In the example of FIG. 14, one of the plurality of display devices 134a-e may be a custom display device 134a specially designed for displaying certain types of displayable sensor information associated with analyte values received from the sensor electronics module 126 (e.g., a numerical value and an arrow, in some examples). In some examples, one of the plurality of display devices 134a-e may be a handheld device 134c, such as a mobile phone based on the Android, IOS operating system or other operating system, a palm-top computer and the like, where handheld device 134c may have a relatively larger display and be configured to display a graphical representation of the continuous sensor data (e.g., including current and historic data). Other display devices can include other hand-held devices, such as a tablet 134d, a smart watch 134b, a medicament delivery device 134e, a blood glucose meter, and/or a desktop or laptop computers.

As alluded to above, because the different display devices 134a-e provide different user interfaces, content of the data packages (e.g., amount, format, and/or type of data to be displayed, alarms, and the like) can be customized (e.g., programmed differently by the manufacture and/or by an end user) for each particular display device and/or display device type. Accordingly, in the example of FIG. 14, one or more of display devices 134a-e can be in direct or indirect wireless communication with the sensor electronics module 126 to enable a plurality of different types and/or levels of display and/or functionality associated with the sensor information, which is described in more detail elsewhere herein.

### Sterilization

The presently disclosed APL-EAB's are configured for sterilization in whole or in part, including aseptic manufacturing and/or packaging. Examples of sterilization methods suitable for the presently disclosed APL-EAB's, include, for example, high energy radiation (UV, e-beam, x-ray), chemical treatment (ethylene oxide, CIDEX OPA™ (0.55% ortho-phthalaldehyde)), or autoclaving.

While certain embodiments of the present disclosure have been illustrated with reference to specific combinations of elements, various other combinations may also be provided without departing from the teachings of the present disclosure. Thus, the present disclosure should not be construed as being limited to the particular exemplary embodiments described herein and illustrated in the Figures, but may also encompass combinations of elements of the various illustrated embodiments and aspects thereof.

1-183. (canceled)

184. An analyte monitoring sensor configured for in vivo measurement of at least one analyte, comprising:
a substrate having a substrate surface;
a co-adsorbate coupled or tethered to the substrate surface and/or an aptamer protective layer encapsulating at least a portion of the substrate surface, the aptamer protective layer permeable to the at least one analyte;
one or more aptamer conjugates associated with at least a portion of the substrate surface and positioned between the aptamer protective layer and the substrate for obtaining measurements related to the at least one analyte in vivo; and
at least one reversible redox moiety coupled to at least one of the one or more aptamer conjugates.

185. The analyte monitoring sensor claim 184, wherein the co-adsorbate is represented as follows: 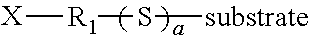
where X is —OH, —NHR1, —NH2 or —SH; where R1 is branched or unbranched acyclic alkyl, substituted or unsubstituted cyclic alkyl, substituted or unsubstituted aryl, substituted or unsubstituted benzyl, substituted or unsubstituted heteroalkyl, or substituted or unsubstituted heterocyclic; and a is 1-3.

186. The analyte monitoring sensor of claim 184, wherein the aptamer protective layer encapsulates the co-adsorbate, wherein the aptamer protective layer is present without the co-adsorbate, or wherein the co-adsorbate is present without the aptamer protective layer.

187. The analyte monitoring sensor claim 184, wherein the aptamer protective layer and/or the co-adsorbate provides one or more of: a free volume allowing reversible conformational change of the one or more aptamer conjugates present therein, the free volume sufficient to provide a signal in the presence of the at least one analyte; an ionic strength; or a localized pH range.

188. The analyte monitoring sensor claim 184, wherein the aptamer protective layer comprises a functionalized polymer.

189. The analyte monitoring sensor claim 184, wherein the aptamer protective layer comprises one or more of polyurethane, polyurea, poly (urethane urea), epoxide, polyolefin, polysiloxane, polyamide, polystyrene, polyacrylate, polyether, polyvinylpyridine, polyvinylpyrrolidone, polyester, polycarbonate, and copolymers thereof.

190. The analyte monitoring sensor claim 184, wherein the one or more aptamer conjugates comprises RNA or DNA nucleotide sequences.

191. The analyte monitoring sensor claim 184, wherein the one or more aptamer conjugates is a glycopeptide antibiotic binding aptamer, is a vancomycin binding aptamer, a neurotransmitter binding aptamer, a dopamine binding aptamer, a L-DOPA binding aptamer, an insulin binding aptamer, a glutamate binding aptamer, a carbohydrate binding aptamer, a triglyceride binding aptamer, fatty acid binding aptamer, a glucose binding aptamer, a glycerol binding aptamer, or beta-hydroxybutyrate binding aptamer.

192. The analyte monitoring sensor claim 184, wherein the reversible redox moiety comprises iron, iridium, ruthenium, osmium, a thiazine dye, ferrocene, methylene blue, or a derivative thereof.

193. A method of extending end of life of an electrochemical aptamer biosensor (EAB), the method comprising:
associating at least one aptamer conjugate to a surface of a conductive substrate, the at least one aptamer conjugate comprising a reversible redox moiety;
one or more co-adsorbates coupled or tethered to the substrate surface and/or encapsulating the at least one aptamer conjugate in an aptamer protective layer, the at least one aptamer conjugate configured to undergo a reversible confirmation change within the aptamer protective layer in response interaction with an analyte so as to generate a detectable signal;
controlling one or more of: ionic strength, surface phase separation of the aptamer protective layer, and intermolecular interactions between the at least one aptamer conjugate and the aptamer protective layer; and
extending the end-of-life of the electrochemical aptamer sensor.

194. The method of claim 193, wherein the aptamer protective layer encapsulates the co-adsorbate, wherein the aptamer protective layer is present without the co-adsorbate, or wherein the co-adsorbate is present without the aptamer protective layer.

195. The method of claim 193, wherein the aptamer protective layer comprises one or more of polyurethane, polyurea, poly (urethane urea), epoxide, polyolefin, polysiloxane, polyamide, polystyrene, polyacrylate, polyether, polyvinylpyridine, polyvinylpyrrolidone, polyester, polycarbonate, and copolymers thereof.

196. The method of claim 193, wherein the one or more aptamer conjugates comprises RNA or DNA nucleotide sequences.

197. The method of claim 193, wherein the one or more aptamer conjugates is a glycopeptide antibiotic binding aptamer, is a vancomycin binding aptamer, a neurotransmitter binding aptamer, a dopamine binding aptamer, a L-DOPA binding aptamer, an insulin binding aptamer, a glutamate binding aptamer, a carbohydrate binding aptamer, a triglyceride binding aptamer, fatty acid binding aptamer, a glucose binding aptamer, a glycerol binding aptamer, or beta-hydroxybutyrate binding aptamer.

198. The method of claim 193, wherein controlling ionic strength comprises the one or more co-adsorbents being present in an amount capable of modulating or maintaining the ionic strength, wherein the one or more co-adsorbents comprises a zwitterionic betaine group of the following structures: 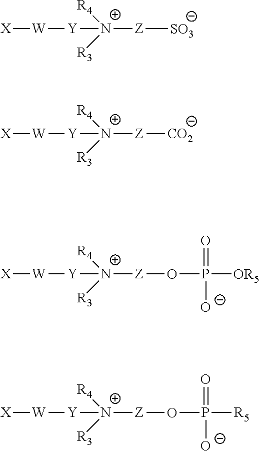
where X is —OH, —NHR1, —NH2, or —SH; where W, Y, and Z are, independently, branched or straight chain alkyl, heteroalkyl, cycloalkyl, cycloheteroalkyl, aryl, or heteroaryl, any of which can be optionally substituted with O, OH, halogen, amido, or alkoxyl; R1 is H, branched or unbranched acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl; and R3, R4, and R5, are independently chosen from acyclic alkyl, substituted or unsubstituted heteroalkyl, substituted or unsubstituted cycloalkyl, substituted or unsubstituted cycloheteroalkyl, substituted or unsubstituted aryl, substituted or unsubstituted benzyl, or substituted or unsubstituted heteroaryl; wherein one or more of R3, R4, R5, W, X, Y, and Z are coupled to the surface of the conductive substrate.

199. The method of claim 193, wherein end-of-life is extended up to one day, 2 days, one week, 2 weeks, 3 weeks, or one month.

200. An aptamer protective layer configured for transcutaneous in vivo continuous online monitoring, the aptamer protective layer comprising a polymer selected from:
a functionalized polymer comprising at least one zwitterionic repeating group;
a functionalized polymer from at least one of a polymerizable zwitterionic monomer structure as follows: 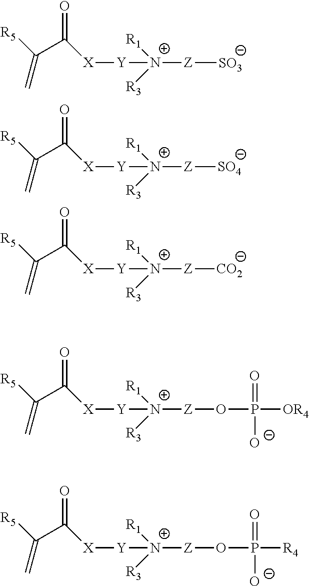
where X is O, NH, or NR₄, Y and Z are, independently, acyclic alkyl, substituted or unsubstituted heteroalkyl, substituted or unsubstituted cycloalkyl, substituted or unsubstituted and of which can be optionally substituted with OH, halogen, or alkoxyl; R₁, R₃, R₄, and R₅ are independently H, alkyl, heteroalkyl, cycloalkyl, cycloheteroalkyl, aryl, or heteroaryl;
a functionalized polymer comprising alkanethiol, phenylthiol, or benzyl thiol groups;
a functionalized polymer comprising alkanethiol groups, phenylthiol groups, or benzyl thiol groups in combination with zwitterionic repeating groups;
a functionalized polymer comprising mercaptoalkanol groups, arylmercaptoalkanol groups, benzylmercaptoalkanol groups, or mixtures thereof;
a functionalized polymer comprising mercaptoalkanol groups, arylmercaptoalkanol groups, benzylmercaptoalkanol groups or mixtures thereof in combination with zwitterionic repeating groups; or
a segmented multiblock polymer.

201. The aptamer protective layer of claim 200, wherein the segment multiblock polymer comprises at least one of polyurethane, polyurea, poly (urethane urea), epoxide, polyolefin, polysiloxane, polyamide, polystyrene, polyacrylate, polyether, polyol, polyvinylpyridine, polyvinylpyrrolidone, polyester, polycarbonate, and copolymers thereof.

202. The aptamer protective layer of claim 200, wherein the aptamer protective layer comprises a segmented multiblock polyurethane polymer or a segmented multiblock polyurethane urea polymer.

203. A method of determining an in vivo concentration of an analyte, the method comprising:
contacting, in vivo, a biological fluid comprising an analyte with an electrochemical aptamer biosensor comprising an aptamer conjugate; a conductive substrate; a co-adsorbate associated with both the aptamer conjugate and the conductive substrate; and/or an aptamer protective layer encapsulating the aptamer conjugate about the conductive substrate, the aptamer protective layer being permeable to an analyte, the electrochemical aptamer biosensor producing a signal upon interaction with the analyte; and
interrogating the conductive substrate or the electrochemical aptamer; and
detecting the signal corresponding to an in vivo concentration of the analyte.

204. The method of claim 203, wherein the aptamer protective layer encapsulates the co-adsorbate, wherein the aptamer protective layer is present without the co-adsorbate, or wherein the co-adsorbate is present without the aptamer protective layer.

205. The method of claim 203, wherein interrogating the conductive substrate comprises a differential measurement technique, the differential measurement technique comprises interrogating the conductive substrate with a first square wave voltammetry (SWV) frequency to obtain a first signal and a second SWV frequency to obtain a second signal, taking the difference between the two signals, and dividing by the average of the two signals to obtain an adjusted signal.

206. The method of claim 205, wherein the interrogating comprises chronoamperometry or cyclic voltammetry.
